(19) United States
(12) Patent Application Publication
(10) Pub. No.: US 2010/0303775 A1
(43) Pub. Date: Dec. 2, 2010

(54) Generation of Genetically Corrected Disease-free Induced Pluripotent Stem Cells

(75) Inventors: ANGEL RAYA, BARCELONA (ES); JUAN ANTONIO BUEREN, MADRID (ES); JUAN CARLOS IZPISUA BELMONTE, LA JOLLA, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: THE SALK INSTITUTE FOR BIOLOGICAL STUDIES, LA JOLLA, CA (US); THE CENTER OF REGENERATIVE MEDICINE, BARCELONA (ES)

(21) Appl. No.: 12/789,375

(22) Filed: May 27, 2010

Related U.S. Application Data
(60) Provisional application No. 61/181,287, filed on May 27, 2009.

(51) Int. Cl. A61K 35/12; C12N 15/09; C12N 5/10; A61P 7/06

(52) U.S. Cl. 424/93.21; 435/455; 435/325; 435/377

(57) ABSTRACT

Methods and compositions for the generation and use of genetically corrected induced pluripotent stem cells are provided.

### CROSS-REFERENCES TO RELATED APPLICATIONS

This application claims the benefit of U.S. Provisional Application No. 61/181,287, filed May 27, 2009, the content of which is incorporated herein by reference in its entirety and for all purposes.

### BACKGROUND OF THE INVENTION

The possibility of reprogramming mature somatic cells to generate iPS cells¹⁻⁵ has opened new perspectives in regenerative medicine. The generation of iPS cells may have a wide range of applications in cell and gene therapy, and could be particularly relevant for the treatment of inherited bone marrow failure (BMF) syndromes, where the progressive decline in hematopoietic stem cell numbers limits the production of peripheral blood cells. In these cases, the generation of disease-free hematopoietic progenitor cells from genetically corrected reprogrammed cells from other tissues may open new therapeutic options not previously considered. Among the different inherited BMF syndromes, Fanconi anemia is the most common⁹. FA is a rare recessive, autosomal or X-linked, chromosomal instability disorder caused by mutations in any of the 13 genes so far identified in the FA/BRCA pathway¹⁰. Cells from these patients display typical chromosomal instability and hypersensitivity to DNA cross-linking agents, characteristics that are used to make the diagnosis of FA¹¹. Most FA patients develop BMF, being the cumulative incidence of 90% by 40 years of age¹². Additionally, FA patients are prone to develop malignancies, principally acute myeloid leukemia and squamous cell carcinomas¹². Currently, the therapy of choice for FA patients is transplantation of hematopoietic grafts from HLA-identical siblings, since the output of transplants from non-related donors is poor^{13,14}. Although the genetic correction of autologous HSCs with integrative vectors may constitute a good therapeutic option for FA patients, gene therapy trials conducted so far have not been clinically successful^{15,16}. The paucity of hematopoietic stem cells in the bone marrow of FA patients¹⁶⁻¹⁸ not only accounts for the BMF occurring in FA patients¹², but also constitutes one of the main factors limiting the efficacy of FA gene therapy^{15,16}. The generation of genetically corrected FA-specific iPS cells by the reprogramming of non-hematopoietic somatic cells would result in the production of large numbers of autologous hematopoietic stem cells that may be used to restore the hematopoietic function in these patients. It is shown herein that somatic cells from Fanconi anemia (FA) patients, upon correction of the genetic defect, can be reprogrammed to pluripotency to generate patient-specific iPS cells. These cell lines appear indistinguishable from human embryonic stem cells and iPS cells from healthy individuals in colony morphology, growth properties, expression of pluripotency-associated transcription factors and surface markers, and differentiation potential in vitro and in vivo. Most importantly, it is demonstrated that corrected FA-specific iPS cells can give rise to hematopoietic progenitors of the myeloid and erythroid lineages that are phenotypically normal, i.e. disease-free. These data offer proof-of-concept that iPS cell technology can be used for the generation of disease-corrected, patient-specific cells with potential value for cell therapy applications.

### BRIEF SUMMARY OF THE INVENTION

Provided herein are, inter alia, highly efficient methods and compositions for making and using genetically corrected induced pluripotent stem cells. The genetically corrected induced pluripotent stem cells may be generated through genetic correction and reprogramming of a non-pluripotent genetically diseased cell.

In one aspect, a method for preparing a genetically corrected induced pluripotent stem cell is provided. The method includes transfecting a genetically diseased non-pluripotent cell with a nucleic acid encoding a disease-correcting gene to form a genetically corrected non-pluripotent cell. The genetically corrected non-pluripotent cell is transfected with a nucleic acid encoding an OCT4 protein, a nucleic acid encoding a SOX2 protein, a nucleic acid encoding a KLF4 protein and a nucleic acid encoding a cMYC protein to form a genetically corrected transfected non-pluripotent cell. The genetically corrected transfected non-pluripotent cell is allowed to divide thereby forming the genetically corrected induced pluripotent stem cell.

In another aspect, a method for preparing a genetically corrected induced pluripotent stem cell is provided. The method includes transfecting a genetically diseased non-pluripotent cell with a nucleic acid encoding an OCT4 protein, a nucleic acid encoding a SOX2 protein, a nucleic acid encoding a KLF4 protein and a nucleic acid encoding a cMYC protein to form a transfected genetically diseased non-pluripotent cell. The transfected genetically diseased non-pluripotent cell is allowed to divide thereby forming a genetically diseased induced pluripotent stem cell. And the genetically diseased induced pluripotent stem cell is transfected with a nucleic acid encoding a disease-correcting gene to form the genetically corrected induced pluripotent stem cell.

In another aspect, a genetically corrected induced pluripotent stem cell is prepared according to the methods provided herein.

In another aspect, a method for producing a genetically corrected somatic cell from a genetically diseased mammal is provided. The method includes contacting a genetically corrected induced pluripotent stem cell with cellular growth factors and allowing the genetically corrected induced pluripotent stem cell to divide, thereby forming the genetically corrected somatic cell.

In another aspect, a method of treating a mammal in need of tissue repair is provided. The method includes administering a genetically corrected induced pluripotent stem cell to the mammal and allowing the genetically corrected induced pluripotent stem cell to divide and differentiate into somatic cells in the mammal, thereby providing tissue repair in the mammal.

In one aspect, a genetically diseased non-pluripotent cell in including a nucleic acid encoding a disease-correcting gene, a nucleic acid encoding an OCT4 protein, a nucleic acid encoding a SOX2 protein, a nucleic acid encoding a KLF4 protein and a nucleic acid encoding a cMYC protein is provided.

### BRIEF DESCRIPTION OF THE DRAWINGS

FIG. 1: Derivation of patient-specific induced pluripotent stem cells from Fanconi anemia patients. FIGS. 1a-1f: Successful reprogramming of genetically corrected primary dermal fibroblasts (FIG. 1a) derived from patient FA90. FIG. 1b: Colony of iPS cells from the cFA90-44-14 line grown on Matrigel-coated plated showing hES cell-like morphology. FIGS. 1c-1f: The same iPS cell line shows strong AP staining (FIG. 1c) and expression of the transcription factors OCT4 (FIG. 1d), SOX2 (FIG. 1e) and NANOG (FIG. 10 and the surface markers SSEA3 (FIGS. 1d-e) and SSEA4 (FIG. 10. FIG. 1g: Genetically corrected fibroblasts from patient FA404. FIG. 1h: Colony of iPS cells from the cFA404-FiPS4F1 line grown on feeder cells displaying typical hES cell morphology. FIGS. 1i-1l: The same iPS cell line shows strong AP staining (FIG. 1i) and expression of the pluripotency-associated transcription factors OCT4 (FIG. 1j), SOX2 (FIG. 1k) and NANOG (FIG. 1l) and surface markers SSEA3 (FIG. 1j), SSEA4 (FIG. 1k) and TRA1-80 (FIG. 1l). Cell nuclei were counterstained with DAPI in FIGS. 1d-1f and 1j-1l. Scale bar, 100 μm (FIGS. 1a, 1c-1g, 1i-1l) and 250 μm (FIGS. 1b, 1h).

FIG. 2: Molecular characterization of FA patient-specific iPS cell lines. FIG. 2a: PCR of genomic DNA to detect integration of the indicated retroviral transgenes in the patient-specific iPS cell lines cFA90-44-14 and cFA404-FiPS4F1. Genetically corrected fibroblasts (Fibr.) from patient FA404 prior to reprogramming were used as negative control. FIGS. 2b-2c: Quantitative RT-PCR analyses of the expression levels of retroviral-derived reprogramming factors (FIG. 2b) and of total expression levels of reprogramming factors and pluripotency-associated transcription factors (FIG. 2c) in the indicated patients' fibroblasts (fibr.) and patient-specific iPS cell lines. hES cells (ES[4]) and partially-silenced iPS cells (KiPS4F3) are included as controls Transcript expression levels are plotted relative to GAPDH expression. FIGS. 2d-2g: Colony of cFA90-44-14 iPS cells showing high levels of endogenous NANOG expression (FIGS. 2e, 2d) and absence of FLAG immunoreactivity (FIGS. 2f, 2d). Cell nuclei were counterstained with DAPI (FIGS. 2g, 2d). FIG. 2h: Bisulfite genomic sequencing of the OCT4 and NANOG promoters showing demethylation in the patient-specific iPS cell lines cFA90-44-14 and cFA404-KiPS4F3, compared to patient's fibroblasts. Open and closed circles represent unmethylated and methylated CpGs, respectively, at the indicated promoter positions. Scale bar, 100 μm. Histograms in FIGS. 2b-2c depict data in the order: cFA90 fibr., cFA90-44-1, cFA90-44-11, cFA90-44-14, cFA90-44-21, cFA404 fibr., cFA404-KIPS4F1, cFA404-KIPS4F3, cFA404-KIPS4F6, cFA404-FIPS4F1, cFA404-FiPS4F2, ES(4) and KIPS4F3.

FIG. 3: Pluripotency of FA patient-specific iPS cells. FIGS. 3a-3c: In vitro differentiation experiments of cFA404-FiPS4F2 iPS cells reveal their potential to generate cell derivatives of all three primary germ cell layers. Immunofluorescence analyses show expression of markers of FIG. 3a, endoderm (α-fetoprotein; FoxA2), FIG. 3b, neuroectoderm (TuJ1; GFAP), and mesoderm (α-actinin) FIGS. 3d-3f: Injection of cFA90-44-14 iPS cells under the skin of immunocompromised mice results in the formation of teratomas containing structures that represent the 3 main embryonic germ layers. Endoderm derivatives (FIGS. 3d-3e) include glandular structures that stain positive for endoderm markers (α-fetoprotein); ectoderm derivatives (FIG. 3e) include structures that stain positive for neuroectoderm markers (TuJ1); mesoderm derivatives (FIG. 3f) include structures that stain positive for muscle markers (α-actinin). All images are from the same tumor. Scale bar, 100 μm (a, b, d, e) and 25 μm (c, f).

FIG. 4: Functional FA pathway in patient-specific iPS cell lines. FIG. 4a: Western blot analysis of FANCA in protein extracts from the indicated cell lines, showing expression of FANCA in FA patient-specific iPS cells. The expression of vinculin was used as loading control. FIG. 4b: FANCD2 fails to relocate to UVC radiation-induced stalled replication forks, visualized by immunofluorescence with antibodies against cyclobutane pyrimidine dimers (CPD), in fibroblasts from patient FA404, while it shows normal accumulation to damaged sites in wild-type fibroblasts (control), corrected fibroblasts (cFA404) or FA-iPS-derived cells (cFA404-FiPS4F2). FIG. 4c: Western blot analysis of FANCA in protein extracts from untransduced cFA404-KiPS4F3 cells or 6 days after transduction with lentiviruses expressing scramble shRNA (Control) or the indicated FANCA-shRNAs. The expression of vinculin was used as loading control. Values at the bottom represent FANCA expression levels measured by densitometry quantification normalized by vinculin expression and referred to untransduced cFA404-KiPS4F3 cells. FIG. 4d: Alkaline phosphatase staining of cFA404-KiPS4F3 cells 1 passage after being transduced with lentiviruses expressing scramble shRNA (Control) or the indicated FANCA-shRNAs, 1 week after seeding. FIG. 4e: Mitotic index values in cFA404-FiPS4F2-derived cells transfected with scramble (Control) or FANCA siRNAs and incubated in the absence or in the presence of diepoxybutane (DEB). The inset shows FANCA depletion induced by FANCA siRNAs in these experiments, as visualized by Western blot using vinculin as loading control.

FIG. 5: Generation of disease-free hematopoietic progenitors from patient-specific iPS cell lines. FIG. 5a: Expression of CD34 and CD45 markers in iPS cells subjected to hematopoietic differentiation. FIGS. 5b-5c: Representative erythroid (BFU-E) and myeloid (CFU-GM) colonies generated 14 days after the incubation of iPS-derived CD34⁺ cells in semisolid cultures. FIG. 5d: The myeloid nature of CFU-GM colonies was confirmed by the co-expression of the CD33 and CD45 markers in CFU-GM colonies. FIG. 5e: Total number of colony-forming cells (CFC) generated in the absence and the presence of 10 nM mitomycin C (MMC) from CD34⁺ cells derived from the indicated FA-iPS cell lines. For comparison, clonogenic assays were also performed using hematopoietic progenitors from healthy donors (purified CD34⁺ cord blood cells from 2 independent donors, CB CD34⁺; and mononuclear bone marrow cells, BM MNC), from a FA patient, and from CD34⁺ cells derived from control human pluripotent stem cells, including ES[2] cells (hES) and KiPS4F1 cells (KiPS). FIG. 5f: Immunofluorescence analysis showing FANCD2 foci in mitomycin C-treated CD34⁺ cells derived from FA-iPS cells (line cFA90-44-14).

FIG. 6: Derivation of self-renewing cells from human fibroblasts. Control human fibroblasts were infected with retroviruses encoding OCT4, SOX2, KLF4, and c-MYC and selected for growth in hES cell medium in the presence of inhibitors PD0325901 and CT99021. FIGS. 6a-6b: Defined colonies of tightly packed cells appearing after 20 d (FIG. 6a) and 30d (FIG. 6b). FIG. 6c: Cells of line T1-4F#14 at passage 10 grown on feeders, displaying mouse ES cell-like colony morphology. FIG. 6d: Injection of T1-4F#14 cells into the testis of immunocompromised mice gave rise to homogeneous tumors composed of undifferentiated cells, not resembling teratomas (FIG. 6d′ is a magnification of the area boxed in FIG. 6d). FIG. 6e: PCR on genomic DNA of T1-4F#14 cells only detected integration of the cMYC transgene.

FIG. 7: Normal karyotype of FA patient specific iPS cells. G-banding karyotype analyses of cFA90-44-14 cells at passage 43 and cFA404-KiPS4F3 cells at passage 24 reveal normal karyotype of FA patient-specific iPS cells.

FIG. 8: Characterization of additional iPS cell lines derived from patient FA90. Immunofluorescence analyses of the expression of the pluripotency-associated transcription factors OCT4, SOX2, and NANOG and surface markers SSEA3, SSEA4, and TRA1-60 in colonies of clonal iPS cell lines derived from corrected fibroblasts of patient FA90.

FIG. 9: Characterization of additional iPS cell lines derived from patient FA404. AP staining (top row) and immunofluorescence analyses of the expression of the pluripotency-associated transcription factors OCT4, SOX2, and NANOG and surface markers SSEA3, SSEA4, and TRA1-60 in colonies of clonal iPS cell lines derived from corrected fibroblasts of patient FA404.

FIG. 10: Characterization of iPS cell lines derived from patient FA431. FIG. 10a: Genetically corrected fibroblasts from patient FA431. FIGS. 10b-10f: iPS cells generated by reprogramming fibroblasts from patient FA431 transduced with FANCD2-expressing lentiviruses (line cFA431-44-1) grow as hES-like colonies (FIG. 10b), stain positive for AP activity (FIG. 10c), and express the pluripotency-associated transcription factors OCT4 (FIG. 10d), SOX2 (FIG. 10e), and NANOG (FIG. 10f) and surface markers SSEA3 (FIG. 10d), TRA1-81 (FIG. 10e), and TRA1-60 (FIG. 10f). FIG. 10g: AP staining of iPS-like colonies of lines generated from unmodified (FA431-44-1) or genetically corrected (cFA431-44-1) fibroblasts 5 days after passage 2 (top images) and 15 (bottom left) or 7 (bottom right) days after passage 3.

FIG. 11: Retroviral integrations in iPS cell lines generated from corrected FA fibroblasts. PCR on genomic DNA from the indicated iPS cell lines showing integration of all 4 retroviruses.

FIG. 12: In vitro differentiation ability of additional FA patient specific iPS cell lines. Immunofluorescence analyses of differentiation markers representing the 3 main embryonic germ layers, endoderm (α-fetoprotein; FoxA2), ectoderm (TuJ1; tyroxine hydroxilase, TH; Glial fibrillary acidic protein, GFAP), and mesoderm (vimentin, α-actinin), in in vitro differentiation assays of the indicated iPS cell lines.

FIG. 13: Teratoma formation of an additional FA patient specific iPS cell line. Injection of cFA404-KiPS4F1 cells into the testis of immunocompromised mice induced the formation of complex teratomas comprising structures derived from the 3 main embryonic germ layers. Endoderm derivatives (top row) included columnar epithelium and structures that stained positive for endoderm markers (α-fetoprotein and FoxA2); ectoderm derivatives (middle row) included pigmented epithelium, neural rosettes and structures that stained positive for neuroectoderm markers (TuJ1 and GFAP); mesoderm derivatives (bottom row) included cartilage and structures that stained positive with muscle markers (α-actinin). All images are from the same tumor. Left and middle columns are hematoxylin and eosin staining, right column are immunofluorescence analyses with the indicated antibodies.

FIG. 14: Phenotypic modification of patient FA404 fibroblasts after transduction with lentiviral vectors encoding FANCA. FIG. 14a: Copy number of lentiviruses expressing FANCA-IRES-EGFP integrated in the genome of the indicated cell lines. *: Represents the average number of lentiviral integrations in non-clonal transduced fibroblasts. **: Copy number value was slightly lower than 2 because of contamination with feeder cells. FIG. 14b: Prior to reprogramming, FA fibroblasts were transduced with FANCA-IRES-EGFP LVs. The analysis of EGFP expression by flow cytometry (histogram panel of FIG. 14b) indicated that 35-50% of the transduced cells were EGFP-positive.

FIG. 15: Functional FA pathway in FAiPS derived cells. FANCD2 fails to relocate to hydroxyurea-induced stalled and broken replication forks (marked by γ-H2AX foci) in FANCA deficient fibroblasts from patient FA404, while it forms normal co-localizing foci in wild type fibroblasts (control), corrected FA fibroblasts (cFA404) or FA-iPS-derived fibroblast-like cells (cFA404-FiPS4F2).

FIG. 16: Derivation of FA patient specific iPS cells without cMYC. FIGS. 16a-16d: Successful reprogramming in the absence of c-MYC retroviruses of genetically-corrected primary epidermal keratinocytes derived from patient FA404. cFA404-KiPS3F1 cells show expression of the transcription factors OCT4 (FIG. 16a), SOX2 (FIG. 16b) and NANOG (FIG. 16c) and the surface markers SSEA3 (FIG. 16a), SSEA4 (FIG. 16b), and TRA1-60 (FIG. 16c), strong AP staining (FIG. 16d). FIGS. 16e-16g: In vitro differentiation of cFA404-KiPS3F1 cells toward endoderm (FIG. 16e, α-fetoprotein; FoxA2) and ectoderm (FIG. 16f, TuJ1) derivatives. Hematopoietic progenitor cells (mesoderm derivatives) at day 10 of differentiation (FIG. 16g).

FIG. 17: Retroviral integrations of reprogramming factors in FA patient-specific iPS cells. Southern blotting to analyze the number of retroviral integrations in the genome of the indicated FA patient-specific iPS cell lines. Genomic DNA digested with the indicated restriction enzymes was blotted and hybridized with probes specific to the reprogramming factors. Genetically corrected fibroblasts from patient FA404 (cFA404 fibr.) were used as control for endogenous bands, marked by asterisks on the left of the blot. Retroviral integrations are indicated by arrowheads. Note the absence of c-MYC integrations in cFA404-KiPS3F1 cells.

### DETAILED DESCRIPTION OF THE INVENTION

### I. Definitions

The following definitions are provided to facilitate understanding of certain terms used frequently herein and are not meant to limit the scope of the present disclosure.

“Nucleic acid” refers to deoxyribonucleotides or ribonucleotides and polymers thereof in either single- or double-stranded form, and complements thereof.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “identical” or percent “identity,” in the context of two or more nucleic acids, refer to two or more sequences or subsequences that are the same or have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The phrase “stringent hybridization conditions” refers to conditions under which a probe will hybridize to its target sequence, typically in a complex mixture of nucleic acids, but to not other sequences. Stringent conditions are sequence-dependent and will be different in different circumstances. Longer sequences hybridize specifically at higher temperatures. An extensive guide to the hybridization of nucleic acids is found in Tijssen, TECHNIQUES IN BIOCHEMISTRY AND MOLECULAR BIOLOGY—HYBRIDIZATION WITH NUCLEIC PROBES, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

A variety of methods of specific DNA and RNA measurement that use nucleic acid hybridization techniques are known to those of skill in the art (see, Sambrook, supra). Some methods involve electrophoretic separation (e.g., Southern blot for detecting DNA, and Northern blot for detecting RNA), but measurement of DNA and RNA can also be carried out in the absence of electrophoretic separation (e.g., by dot blot).

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman® and molecular beacon probes can be used to monitor amplification reaction products, e.g., in real time.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA.

The words “protein”, “peptide”, and “polypeptide” are used interchangeably to denote an amino acid polymer or a set of two or more interacting or bound amino acid polymers.

The term “gene” refers to the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

A “deletion” is defined as a change in either nucleotide or amino acid sequence in which one or more nucleotides or amino acid residues, respectively, are absent.

An “insertion” or “addition” as used herein, is a change in a nucleotide or amino acid sequence which has resulted in the addition of one or more nucleotides or amino acid residues, respectively, as compared to naturally occurring sequences.

A “substitution” results from the replacement of one or more nucleotides or amino acids by different nucleotides or amino acids, respectively.

A “variant” in regard to amino acid sequences is used herein to indicate an amino acid sequence that differs by one or more amino acids from another, usually related amino acid. The variant may have “conservative” changes, wherein a substituted amino acid has similar structural or chemical properties (e.g. replacement of leucine with isoleucine). A variant may have “non-conservative” changes, e.g., replacement of a glycine with a tryptophan. Similar minor variations may also include amino acid deletions or insertions (i.e. additions), or both.

A “locus” as used herein is a fixed position on a chromosome that may be occupied by one or more genes. The locus of a gene on a chromosome is determined by its linear order relative to the other genes on that chromosome. A variant of the DNA sequence at a given locus is called “allele”.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “transfection” or “transfecting” is defined as a process of introducing nucleic acid molecules to a cell by non-viral and viral-based methods. For non-viral methods of transfection any appropriate transfection method that does not use viral DNA or viral particles as a delivery system to introduce the nucleic acid molecule into the cell is useful in the methods described herein. Exemplary transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral based methods of transfection any useful viral vector may be used in the methods described herein. Examples for viral vectors include, but are not limited to retroviral, adenoviral, lentiviral and adeno-associated viral vectors.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion the gene is positioned between two transposon linker sequences that allow insertion into the host genome as well as subsequent excision.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

A “cell culture” is a population of cells residing outside of an organism. These cells are optionally primary cells isolated from a cell bank, animal, or blood bank, or secondary cells that are derived from one of these sources and have been immortalized for long-lived in vitro cultures.

A “stem cell” is a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

The term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells.

“Pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

An “induced pluripotent stem cell” refers to a pluripotent stem cell artificially derived from a non-pluripotent cell. A non-pluripotent cell can be a cell of lesser potency to self-renew and differentiate than a pluripotent stem cell. Cells of lesser potency can be, but are not limited to, somatic stem cells, tissue specific progenitor cells, primary or secondary cells. Without limitation, a somatic stem cell can be a hematopoietic stem cell, a mesenchymal stem cell, an epithelial stem cell, a skin stem cell or a neural stem cell. A tissue specific progenitor refers to a cell devoid of self-renewal potential that is committed to differentiate into a specific organ or tissue. A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and has been immortalized for long-lived in vitro cell culture.

The term “reprogramming” refers to the process of dedifferentiating a non-pluripotent cell into a cell exhibiting pluripotent stem cell characteristics.

The term “treating” means ameliorating, suppressing, eradicating, and/or delaying the onset of the disease being treated.

### II. Methods of Preparing Genetically Corrected Induced Pluripotent Stem Cells

In one aspect, a method for preparing a genetically corrected induced pluripotent stem cell is provided. The method includes transfecting a genetically diseased non-pluripotent cell with a nucleic acid encoding a disease-correcting gene to form a genetically corrected non-pluripotent cell. The genetically corrected non-pluripotent cell is transfected with a nucleic acid encoding an OCT4 protein, a nucleic acid encoding a SOX2 protein, a nucleic acid encoding a KLF4 protein and a nucleic acid encoding a cMYC protein to form a genetically corrected transfected non-pluripotent cell. The genetically corrected transfected non-pluripotent cell is allowed to divide thereby forming the genetically corrected induced pluripotent stem cell.

A “genetically corrected induced pluripotent stem cell” refers to an induced pluripotent stem cell that originates from a genetically diseased non-pluripotent cell and has been corrected for a genetic defect. The genetically diseased non-pluripotent cell includes a genetic defect of a single gene or allele. Through correction of the genetic defect before reprogramming of the non-pluripotent cell a genetically corrected induced pluripotent stem cell is generated. The genetic defect may form the basis for a monogenic disease and includes, but is not limited to base pair deletions, insertions or mutations in a gene. Monogenic diseases include disorders that result from defects in a single gene and can be dominant, recessive or x-linked. Recessive monogenic diseases are characterized by a defect of both copies of a gene. Dominant monogenic diseases involve defects in only one gene copy. X-linked monogenic diseases are disorders that are linked to defective genes on the X chromosome. Examples for monogenic disease are severe combined immunodeficiency disease, thalassaemia, sickle cell anemia, Fanconi anaemia, haemophilia A, haemophilia B, cystic fibrosis, α1-antitrypsin deficiency, Canavan disease, muscular dystrophy, adenosine deaminase deficiency, Tay Sachs disease, Fragile X chromosome, Huntington's disease, Gaucher's disease, Hurler's disease, von Recklinghausen's disease, familial hypercholesterolemia, von Willebrand disease, Congenital leptin deficiency, Congenital neurogenic diabetes insipidus, Fabry disease, and Pompe disease.

A genetically diseased non-pluripotent cell may be corrected by introducing a disease-correcting gene. A disease-correcting gene is a non-defective version of the defective gene causing the disease. The disease correcting gene may be introduced to the genetically diseased non-pluripotent cell according to the transfection methods described herein. The expression of the disease-correcting gene generates a non-diseased cell thereby forming a genetically corrected non-pluripotent cell.

An “OCT4 protein” as referred to herein includes any of the naturally-occurring forms of the Octomer 4 transcription factor, or variants thereof that maintain Oct4 transcription factor activity (e.g. within at least 50%, 80%, 90%, 95%, 96%, 97%, 98%, 99% or 100% activity compared to Oct4). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Oct4 polypeptide (e.g. SEQ ID NO:1, SEQ ID NO:2 or SEQ ID NO:3). In other embodiments, the Oct4 protein is the protein as identified by the NCBI reference gi:42560248 corresponding to isoform 1 (SEQ ID NO:1), and gi:116235491 and gi:291167755 corresponding to isoform 2 (SEQ ID NO:2 and SEQ ID NO:3).

A “SOX2 protein” as referred to herein includes any of the naturally-occurring forms of the Sox2 transcription factor, or variants thereof that maintain Sox2 transcription factor activity (e.g. within at least 50%, 80%, 90%, 95%, 96%, 97%, 98%, 99% or 100% activity compared to Sox2). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Sox2 polypeptide (e.g. SEQ ID NO:4). In other embodiments, the Sox2 protein is the protein as identified by the NCBI reference gi:28195386 (SEQ ID NO:4).

A “KLF4 protein” as referred to herein includes any of the naturally-occurring forms of the KLF4 transcription factor, or variants thereof that maintain KLF4 transcription factor activity (e.g. within at least 50%, 80%, 90%, 95%, 96%, 97%, 98%, 99% or 100% activity compared to KLF4). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KLF4 polypeptide (e.g. SEQ ID NO:5). In other embodiments, the KLF4 protein is the protein as identified by the NCBI reference gi:194248077 (SEQ ID NO:5).

A “cMYC protein” as referred to herein includes any of the naturally-occurring forms of the cMyc transcription factor, or variants thereof that maintain cMyc transcription factor activity (e.g. within at least 50%, 80%, 90%, 95%, 96%, 97%, 98%, 99% or 100% activity compared to cMyc). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cMyc polypeptide (e.g. SEQ ID NO:6). In other embodiments, the cMyc protein is the protein as identified by the NCBI reference gi:71774083 (SEQ ID NO:6).

Allowing the genetically corrected transfected non-pluripotent cell to divide and thereby forming the genetically corrected induced pluripotent stem cell may include expansion of the genetically corrected transfected non-pluripotent cell after transfection, optional selection for transfected cells and identification of pluripotent stem cells. Expansion as used herein includes the production of progeny cells by a genetically corrected transfected non-pluripotent cell in containers and under conditions well know in the art. Expansion may occur in the presence of suitable media and cellular growth factors. Cellular growth factors are agents which cause cells to migrate, differentiate, transform or mature and divide. They are polypeptides which can usually be isolated from various normal and malignant mammalian cell types. Some growth factors can also be produced by genetically engineered microorganisms, such as bacteria (E. coli) and yeasts. Cellular growth factors may be supplemented to the media and/or may be provided through co-culture with irradiated embryonic fibroblast that secrete such cellular growth factors. Examples of cellular growth factors include, but are not limited to, FGF, bFGF2, and EGF.

Where appropriate the expanding of the genetically corrected transfected non-pluripotent cell may be subjected to a process of selection. A process of selection may include a selection marker introduced into a neural stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected neural stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection maker a toxin may be converted to a non-toxin which no longer inhibits expansion and causes cell death of a genetically corrected transfected non-pluripotent cell. Upon exposure to a toxin a cell lacking a selection marker may be eliminated and thereby precluded from expansion.

Identification of the genetically corrected induced pluripotent stem cell may include, but is not limited to the evaluation of the afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

The genetically diseased non-pluripotent cell may be a mammalian cell. In some embodiments, the genetically diseased non-pluripotent cell is a human cell. In other embodiments, the genetically diseased non-pluripotent cell is a mouse cell.

The disease-correcting gene may encode a polypeptide which upon expression may compensate for the gene defect and restore the status of a non-diseased cell. In some embodiments, the disease-correcting gene encodes a FANCA protein. A “FANCA protein” as referred to herein stands for Fanconi anemia complementation group A and includes any of the naturally-occurring forms of the FANCA protein, or variants thereof that maintain FANCA protein activity (e.g. within at least 50%, 80%, 90%, 95%, 96%, 97%, 98%, 99% or 100% activity compared to FANCA). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring FANCA polypeptide (e.g. SEQ ID NO:7). In other embodiments, the FANCA protein is the protein as identified by the NCBI reference gi: 66880553 (SEQ ID NO:7). In other embodiments, the disease-correcting gene encodes a FANCD2 protein. A “FANCD2 protein” as referred to herein stands for Fanconi anemia complementation group D2 and includes any of the naturally-occurring forms of the FANCD2 protein, or variants thereof that maintain FANCD2 protein activity (e.g. within at least 50%, 80%, 90%, 95%, 96%, 97%, 98%, 99% or 100% activity compared to FANCD2). In some embodiments, variants have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring FANCD2 polypeptide (e.g. SEQ ID NO:8). In other embodiments, the FANCD2 protein is the protein as identified by the NCBI reference gi: 21361861 (SEQ ID NO:8).

The methods described herein may include the introduction of a kinase inhibitor when the genetically corrected transfected non-pluripotent cell is allowed to divide and thereby forms the genetically corrected pluripotent stem cell. A kinase inhibitor is an enzyme inhibitor that specifically blocks the action of one or more protein kinases. Depending on the amino acid being phosphorylated the kinases can be subdivided into serine and threonine kinases, tyrosine kinases and histidine kinases. A kinase inhibitor prevents phosphorylation of such amino acids. Examples of a kinase inhibitor include, but are not limited to monoclonal antibodies, small molecules and organic compounds. The kinase inhibitor may be added to the genetically corrected non-pluripotent cell upon transfection with the nucleic acids encoding an OCT4 protein, a SOX2 protein, a KLF4 protein and a cMYC protein. The kinase inhibitor may be added to the genetically corrected non-pluripotent cell after transfection with the nucleic acids encoding an OCT4 protein, a SOX2 protein, a KLF4 protein and a cMYC protein. In some embodiments, at least one kinase inhibitor is introduced to the genetically corrected transfected non-pluripotent cell of step (iii). In other embodiments, a MEK1 and a GSK3 kinase inhibitor is introduced to the genetically corrected transfected non-pluripotent cell of step (iii).

In another aspect, a method for preparing a genetically corrected induced pluripotent stem cell is provided. The method includes transfecting a genetically diseased non-pluripotent cell with a nucleic acid encoding an OCT4 protein, a nucleic acid encoding a SOX2 protein, a nucleic acid encoding a KLF4 protein and a nucleic acid encoding a cMYC protein to form a transfected genetically diseased non-pluripotent cell. The transfected genetically diseased non-pluripotent cell is allowed to divide thereby forming a genetically diseased induced pluripotent stem cell. And the genetically diseased induced pluripotent stem cell is transfected with a nucleic acid encoding a disease-correcting gene to form the genetically corrected induced pluripotent stem cell.

Allowing the transfected genetically diseased non-pluripotent cell to divide and thereby forming the genetically diseased induced pluripotent stem cell may include expansion of the transfected genetically non-pluripotent cell after transfection, optional selection for transfected cells and identification of pluripotent stem cells. Expansion as used herein includes the production of progeny cells by a genetically corrected transfected non-pluripotent cell in containers and under conditions well know in the art. Expansion may occur in the presence of suitable media and cellular growth factors. Cellular growth factors are agents which cause cells to migrate, differentiate, transform or mature and divide. They are polypeptides which can usually be isolated from various normal and malignant mammalian cell types. Some growth factors can also be produced by genetically engineered microorganisms, such as bacteria (E. coli) and yeasts. Cellular growth factors may be supplemented to the media and/or may be provided through co-culture with irradiated embryonic fibroblast that secrete such cellular growth factors. Examples of cellular growth factors include, but are not limited to, FGF, bFGF2, and EGF.

Where appropriate the expanding of the transfected genetically diseased non-pluripotent cell may be subjected to a process of selection. A process of selection may include a selection marker introduced into a neural stem cell upon transfection. A selection marker may be a gene encoding for a polypeptide with enzymatic activity. The enzymatic activity includes, but is not limited to, the activity of an acetyltransferase and a phosphotransferase. In some embodiments, the enzymatic activity of the selection marker is the activity of a phosphotransferase. The enzymatic activity of a selection marker may confer to a transfected neural stem cell the ability to expand in the presence of a toxin. Such a toxin typically inhibits cell expansion and/or causes cell death. Examples of such toxins include, but are not limited to, hygromycin, neomycin, puromycin and gentamycin. In some embodiments, the toxin is hygromycin. Through the enzymatic activity of a selection maker a toxin may be converted to a non-toxin which no longer inhibits expansion and causes cell death of a genetically corrected transfected non-pluripotent cell. Upon exposure to a toxin a cell lacking a selection marker may be eliminated and thereby precluded from expansion.

Identification of the genetically diseased induced pluripotent stem cell may include, but is not limited to the evaluation of the afore mentioned pluripotent stem cell characteristics. Such pluripotent stem cell characteristics include without further limitation, the expression or non-expression of certain combinations of molecular markers. Further, cell morphologies associated with pluripotent stem cells are also pluripotent stem cell characteristics.

The genetically diseased non-pluripotent cell may be a mammalian cell. In some embodiments, the genetically diseased non-pluripotent cell is a human cell. In other embodiments, the genetically diseased non-pluripotent cell is a mouse cell.

The disease-correcting gene may encode a polypeptide which upon expression may compensate for the gene defect and restore the status of a non-diseased cell. In some embodiments, the disease-correcting gene encodes a FANCA protein. In other embodiments, the FANCA protein is the protein as identified by the NCBI reference gi: 66880553. In some embodiments, the disease-correcting gene encodes a FANCD2 protein. In other embodiments, the FANCD2 protein is the protein as identified by the NCBI reference gi: 21361861.

The methods described herein may include the introduction of a kinase inhibitor when the transfected genetically diseased non-pluripotent cell is allowed to divide and thereby forms the genetically diseased pluripotent stem cell. The kinase inhibitor may be added to the genetically diseased non-pluripotent cell upon transfection with the nucleic acids encoding an OCT4 protein, a SOX2 protein, a KLF4 protein and a cMYC protein. The kinase inhibitor may be added to the genetically diseased non-pluripotent cell after transfection with the nucleic acids encoding an OCT4 protein, a SOX2 protein, a KLF4 protein and a cMYC protein. In some embodiments, at least one kinase inhibitor is introduced to the genetically diseased non-pluripotent cell of step (ii). In other embodiments, a MEK1 and a GSK3 kinase inhibitor is introduced to the genetically diseased non-pluripotent cell of step (ii).

The disease correcting gene may be introduced to the genetically diseased pluripotent stem cell according to the transfection methods described herein. The expression of the disease-correcting gene generates the status of a non-diseased cell thereby forming a genetically corrected pluripotent stem cell.

### III. A Genetically Corrected Induced Pluripotent Stem Cell

In one aspect, a genetically corrected induced pluripotent stem cell is prepared according to the methods provided herein.

### IV. Methods for Producing Human Somatic Cells from Genetically Corrected Induced Pluripotent Stem Cells

In another aspect, a method for producing a genetically corrected somatic cell from a genetically diseased mammal is provided. The method includes contacting a genetically corrected induced pluripotent stem cell with cellular growth factors and allowing the genetically corrected induced pluripotent stem cell to divide, thereby forming the genetically corrected somatic cell. Examples for cellular growth factors include, but are not limited to, SCF, GMCSF, FGF, TNF, IFN, EGF, IGF and members of the interleukin family. The genetically corrected induced pluripotent stem cell is prepared in accordance with the methods provided by the present invention. In some embodiments, a genetically diseased non-pluripotent cell is transfected with a nucleic acid encoding a disease-correcting gene to form a genetically corrected non-pluripotent cell. The genetically corrected non-pluripotent cell is transfected with a nucleic acid encoding an OCT4 protein, a nucleic acid encoding a SOX2 protein, a nucleic acid encoding a KLF4 protein and a nucleic acid encoding a cMYC protein to form a genetically corrected transfected non-pluripotent cell. The genetically corrected transfected non-pluripotent cell is allowed to divide thereby forming the genetically corrected induced pluripotent stem cell. In some embodiments, at least one kinase inhibitor is introduced to the genetically corrected transfected non-pluripotent cell of step (iii). In other embodiments, a MEK1 and a GSK3 kinase inhibitor is introduced to the genetically corrected transfected non-pluripotent cell of step (iii).

In other embodiments, a genetically diseased non-pluripotent cell is transfected with a nucleic acid encoding an OCT4 protein, a nucleic acid encoding a SOX2 protein, a nucleic acid encoding a KLF4 protein and a nucleic acid encoding a cMYC protein to form a transfected genetically diseased non-pluripotent cell. The transfected genetically diseased non-pluripotent cell is allowed to divide thereby forming a genetically diseased induced pluripotent stem cell. The genetically diseased induced pluripotent stem cell is transfected with a nucleic acid encoding a disease-correcting gene to form the genetically corrected induced pluripotent stem cell. In some embodiments, at least one kinase inhibitor is introduced to the genetically diseased non-pluripotent cell of step (ii). In other embodiments, a MEK1 and a GSK3 kinase inhibitor is introduced to the genetically diseased non-pluripotent cell of step (ii).

In another aspect, a method of treating a mammal in need of tissue repair is provided. The method includes administering a genetically corrected induced pluripotent stem cell to the mammal and allowing the genetically corrected induced pluripotent stem cell to divide and differentiate into somatic cells in the mammal, thereby providing tissue repair in the mammal. The genetically corrected induced pluripotent stem cell is prepared in accordance with the methods provided by the present invention. In some embodiments, a genetically diseased non-pluripotent cell is transfected with a nucleic acid encoding a disease-correcting gene to form a genetically corrected non-pluripotent cell. The genetically corrected non-pluripotent cell is transfected with a nucleic acid encoding an OCT4 protein, a nucleic acid encoding a SOX2 protein, a nucleic acid encoding a KLF4 protein and a nucleic acid encoding a cMYC protein to form a genetically corrected transfected non-pluripotent cell. The genetically corrected transfected non-pluripotent cell is allowed to divide thereby forming the genetically corrected induced pluripotent stem cell. In some embodiments, at least one kinase inhibitor is introduced to the genetically corrected transfected non-pluripotent cell of step (iii). In other embodiments, a MEK1 and a GSK3 kinase inhibitor is introduced to the genetically corrected transfected non-pluripotent cell of step (iii).

In other embodiments, a genetically diseased non-pluripotent cell is transfected with a nucleic acid encoding an OCT4 protein, a nucleic acid encoding a SOX2 protein, a nucleic acid encoding a KLF4 protein and a nucleic acid encoding a cMYC protein to form a transfected genetically diseased non-pluripotent cell. The transfected genetically diseased non-pluripotent cell is allowed to divide thereby forming a genetically diseased induced pluripotent stem cell. The genetically diseased induced pluripotent stem cell is transfected with a nucleic acid encoding a disease-correcting gene to form the genetically corrected induced pluripotent stem cell. In some embodiments, at least one kinase inhibitor is introduced to the genetically diseased non-pluripotent cell of step (ii). In other embodiments, a MEK1 and a GSK3 kinase inhibitor is introduced to the genetically diseased non-pluripotent cell of step (ii).

### V. Non-Pluripotent Cells

Provided herein are genetically diseased non-pluripotent cells useful as intermediates in making genetically corrected induced pluripotent stem cells.

In one aspect, a genetically diseased non-pluripotent cell in including a nucleic acid encoding a disease-correcting gene, a nucleic acid encoding an OCT4 protein, a nucleic acid encoding a SOX2 protein, a nucleic acid encoding a KLF4 protein and a nucleic acid encoding a cMYC protein is provided. In some embodiments, the genetically diseased non-pluripotent cell includes at least one kinase inhibitor. In other embodiments, the genetically diseased non-pluripotent cell includes a MEK1 and a GSK3 kinase inhibitor. In some embodiments, the disease-correcting gene is encoding a FANCA protein. In other embodiments, the disease-correcting gene is encoding a FANCD2 protein.

### EXAMPLES

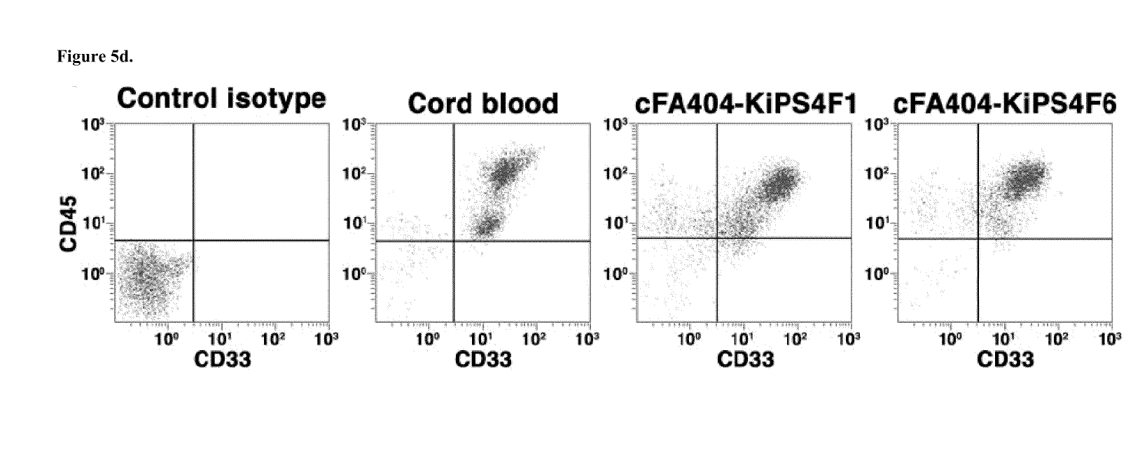

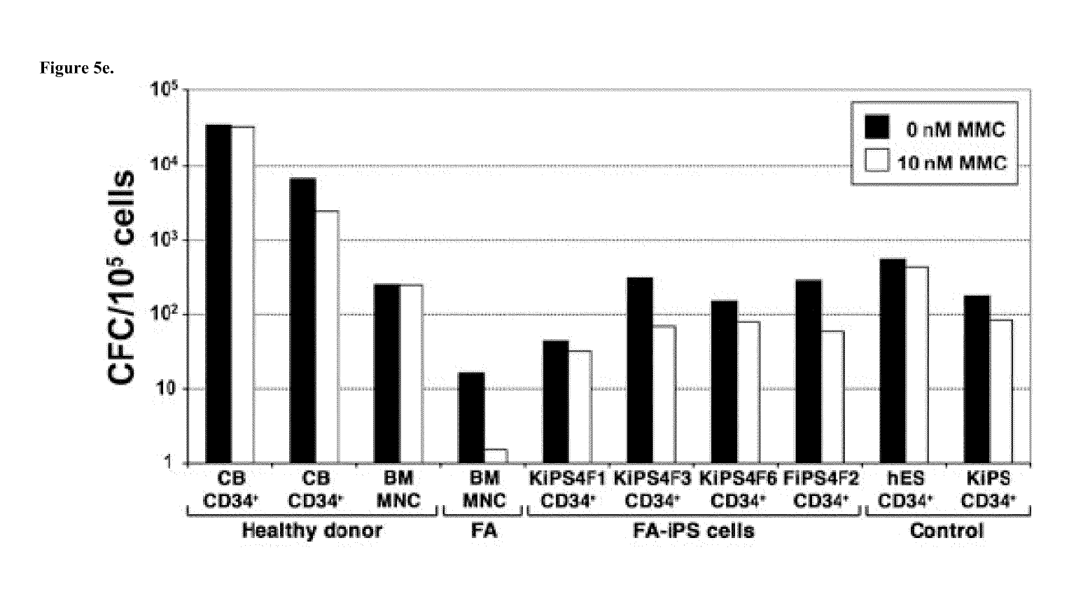

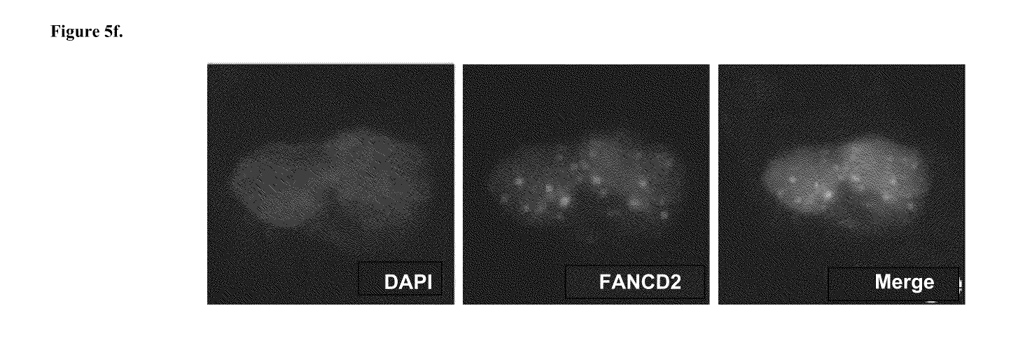

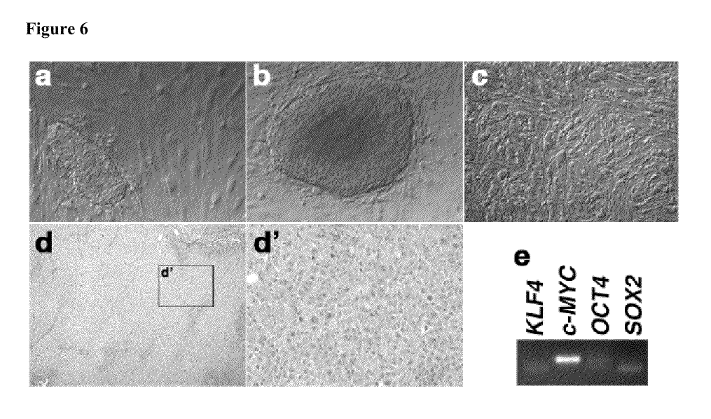

In this study, samples from 6 FA patients were obtained, 4 of which are from the FA-A complementation group (patients FA5, FA90, FA153, and FA404) and 2 from the FA-D2 complementation group (FA430 and FA431). Samples from patients FA5, FA90, FA153, FA430, and FA431 were cryopreserved primary dermal fibroblasts that had undergone an undetermined number of passages. From patient FA404 a skin biopsy was obtained, from which primary cultures of dermal fibroblasts and epidermal keratinocytes were established. Current protocols of induced reprogramming are highly inefficient for human fibroblasts, especially adult human fibroblasts. Successful reprogramming of human adult fibroblasts with retroviruses encoding OCT4, SOX2, KLF4 and c-MYC has been achieved by prior lentiviral transduction with the mouse receptor for retroviruses, co-transduction with hTERT and SV40 large T⁴ or by using VSVg-pseudotyped retroviruses^{6,7}. Even under those conditions, the reprogramming efficiency of human adult fibroblasts is as low as 0.01-0.02%. Similarly, lentiviral delivery of OCT4, SOX2, NANOG, and LIN28 has been reported to reprogram human adult fibroblasts, although at even lower efficiencies (0.001%, ref 8). For this reason, it was first attempted to optimize the reprogramming protocol using primary dermal fibroblasts from a foreskin biopsy of a healthy donor. See FIG. 6. The improved reprogramming protocol consisted of 2 rounds of infection with murine stem cell virus-(MSCV) based retroviruses encoding N-terminal FLAG-tagged versions of OCT4, SOX2, KLF4 and c-MYC, performed 6 days apart. Transduced fibroblasts were passaged after 5 days onto a feeder layer of mitotically-inactivated primary human fibroblasts and then switched to human embryonic stem (hES) cell medium the next day. Also included was a selection step based on the combined inhibition of MEK1 and GSK3 with inhibitors PD0325901 and CT99021 (a combination termed 21 that enhances derivation and growth of mouse ES cells¹⁹) for 1 week, starting 1 week after plating onto feeders.

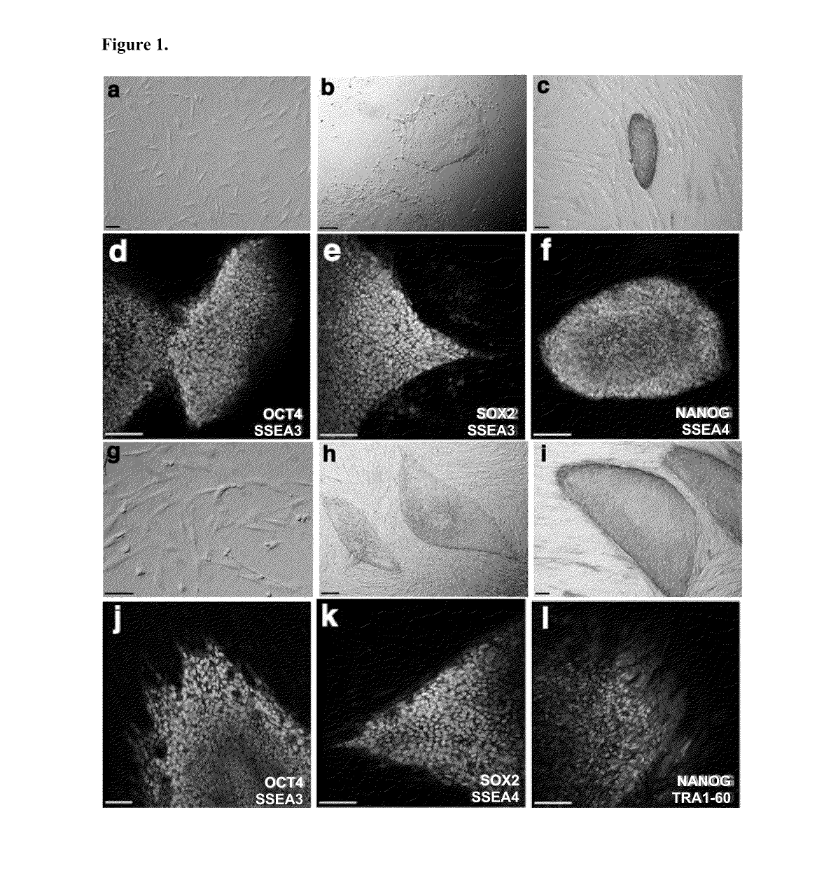

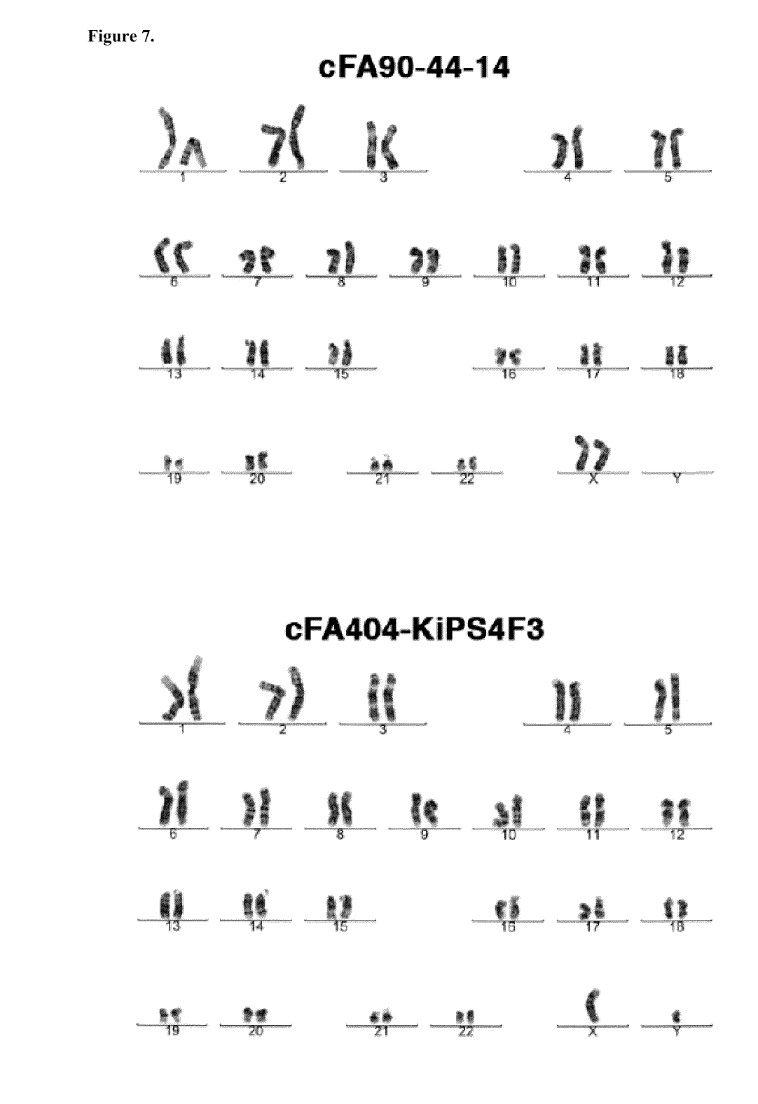

Because of the genetic instability and apoptotic predisposition of FA cells²⁰, skin cells from FA-A and FA-D2 patients were reprogrammed, either directly or after genetic correction with lentiviral vectors encoding FANCA or FANCD2, respectively. It has been previously shown that genetic complementation of human and mouse FA cells with these vectors efficiently corrects the FA phenotype²¹. iPS-like colonies from fibroblasts of patients FAS, FA153 or FA430, either unmodified or corrected, after at least 5 reprogramming attempts were not obtained. Without wishing to be bound by any theory, it is believed that this result is probably owing to the cells having accumulated too many passages and/or karyotypic abnormalities. See Table 1 following. However, from patient FA90 iPS-like colonies were readily obtained when using genetically corrected fibroblasts (FIG. 1a). Overall, 10-15 iPS-like colonies were obtained in each of 3 independent experiments. Of these, 10 colonies were randomly picked, all of which could successfully be expanded on feeder layers or Matrigel-coated plates and grew as flat colonies of tightly packed cells with a high nucleus-to-cytoplasm ratio (FIG. 1b), morphologically indistinguishable from hES cells, and stained strongly positive for alkaline phosphatase (AP) activity (FIG. 1c). Five of these lines (cFA90-44-1, -11, -14, -20, and -21) were selected for further characterization. All of them displayed a normal karyotype (46 XX) at passages 12-16 and could be maintained in culture for, at least, 20 passages. Indeed, cFA90-44-14 had undergone 43 passages without signs of replicative crisis, while maintaining a normal karyotype (FIG. 7).

TABLE 1
Overview of derivation attempts of FA patient specific iPS cell lines.

| Patient | FA | Somatic | | iPS-like | Lines | Lines characterized | | |
|---|---|---|---|---|---|---|---|---|
| ID | group | cell | Attempts | colonies | generated | Markers | In vitro | Terat. |
| FA5 | A | Fibr. | 5 | 0 | 0 | NA | NA | NA |
| FA5 | A | cFibr. | 5 | 0 | 0 | NA | NA | NA |
| FA90 | A | Fibr. | 3 | 0 | 0 | NA | NA | NA |
| FA90 | A | cFibr. | 3 | ~37 | 10 | 5 | 5 | 3 |
| | | | | | cFA90-44-X | | | |
| FA153 | A | Fibr. | 5 | 0 | 0 | NA | NA | NA |
| FA153 | A | cFibr. | 5 | 0 | 0 | NA | NA | NA |
| FA404 | A | Fibr. | 3 | 0 | 0 | NA | NA | NA |
| FA404 | A | cFibr. | 3 | ~30 | 2 | 2 | 2 | 2 |
| | | | | | cFA404-FiPS4FX | | | |
| FA404 | A | Kerat. | 3 | 0 | 0 | NA | NA | NA |
| FA404 | A | cKerat | 3 | ~30 | 3 | 3 | 3 | 3 |
| | | | | | cFA404-KiPS4FX | | | |
| FA430 | D2 | Fibr. | 6 | 0 | 0 | NA | NA | NA |
| FA430 | D2 | cFibr. | 6 | 0 | 0 | NA | NA | NA |
| FA431 | D2 | Fibr. | 3 | ~10 | 2* | 2* | NA | NA |
| FA431 | D2 | cFibr. | 3 | ~10 | 2 | 2 | 2 | NT |
| | | | | | cFA431-44-X | | | |

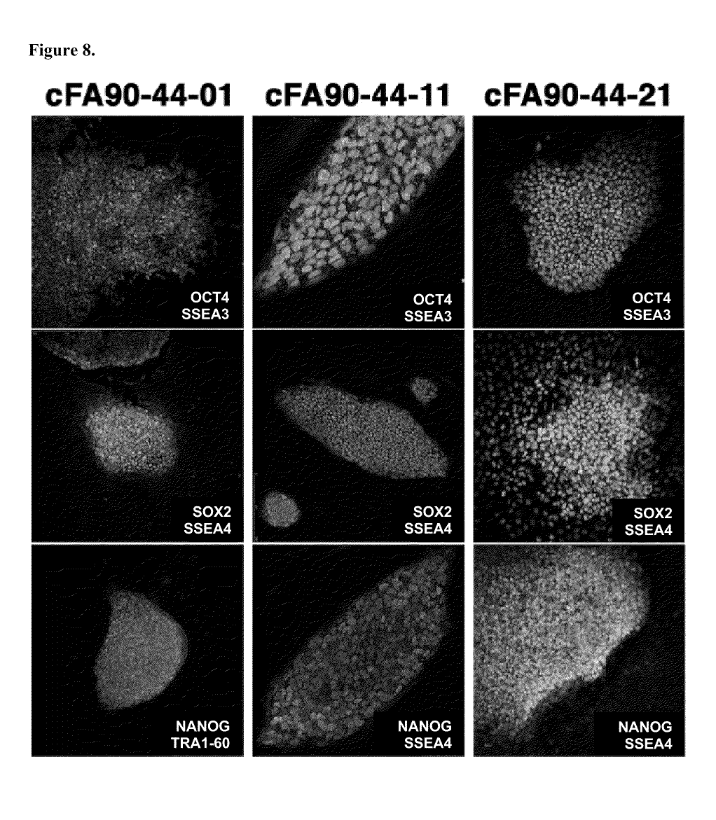

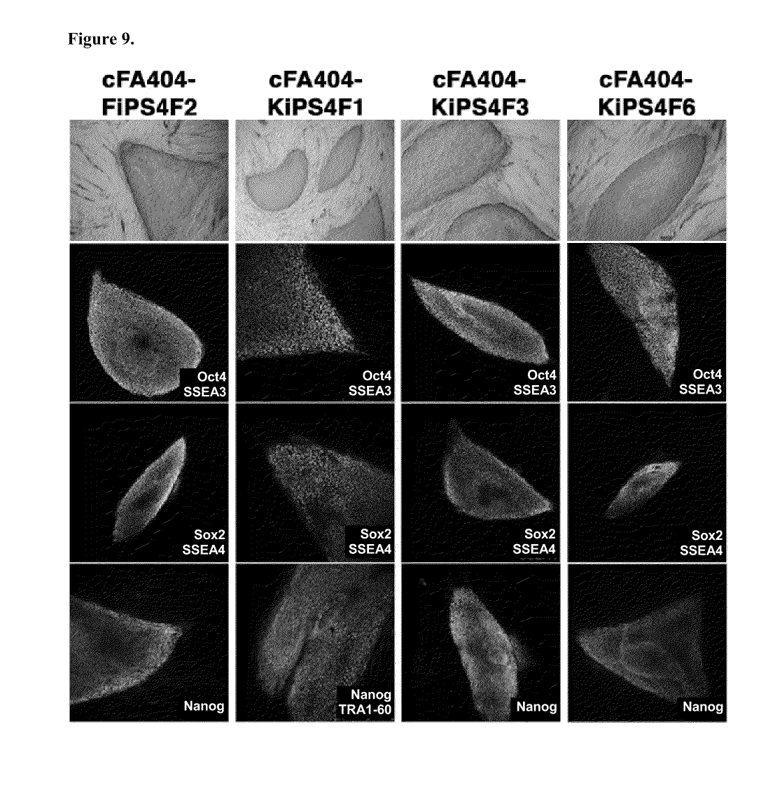

Immunofluorescence analyses of the 5 lines revealed expression of high levels of transcription factors (OCT4, SOX2, NANOG) and surface markers (SSEA3, SSEA4, TRA1-60, TRA1-81) characteristic of pluripotent cells (FIGS. 1d-1f and FIG. 8). These results indicated the successful generation of patient-specific iPS cell lines from fibroblasts of a FA-A patient. In another series of experiments, it was attempted to reprogram somatic cells from another FA-A patient, patient FA404, and obtained similar results. Fibroblasts that had been transduced with lentiviruses encoding FANCA (FIG. 1g) were readily reprogrammed to generate iPS-like cells (FIG. 1h). Two cell lines (cFA404-FiPS4F1 and cFA404-FiPS4F2) were established which were expanded and analyzed in detail. These cell lines displayed typical hES-like morphology and growth characteristics, stained positive for AP activity, and expressed all the pluripotency-associated markers tested (FIGS. 1i-1l and FIG. 9). From patient FA404 primary epidermal keratinocytes, which were attempted to be reprogrammed using an efficient protocol recently set up in the laboratory²² were also derived. iPS cells from keratinocytes transduced with FANCA-expressing lentiviruses were successfully generated, but not from uncorrected keratinocytes. The overall reprogramming efficiency of keratinocytes from patient FA404 was much lower (−20-fold) than that of early-passage primary juvenile keratinocytes from healthy donors²². Nevertheless, the 3 iPS cell lines that were established from these experiments (cFA404-KiPS4F1, -KiPS4F3, and -KiPS4F6) displayed all the main characteristics of bona fide iPS cells and hES cells (FIG. 9) and a normal 46 XY karyotype (FIG. 7).

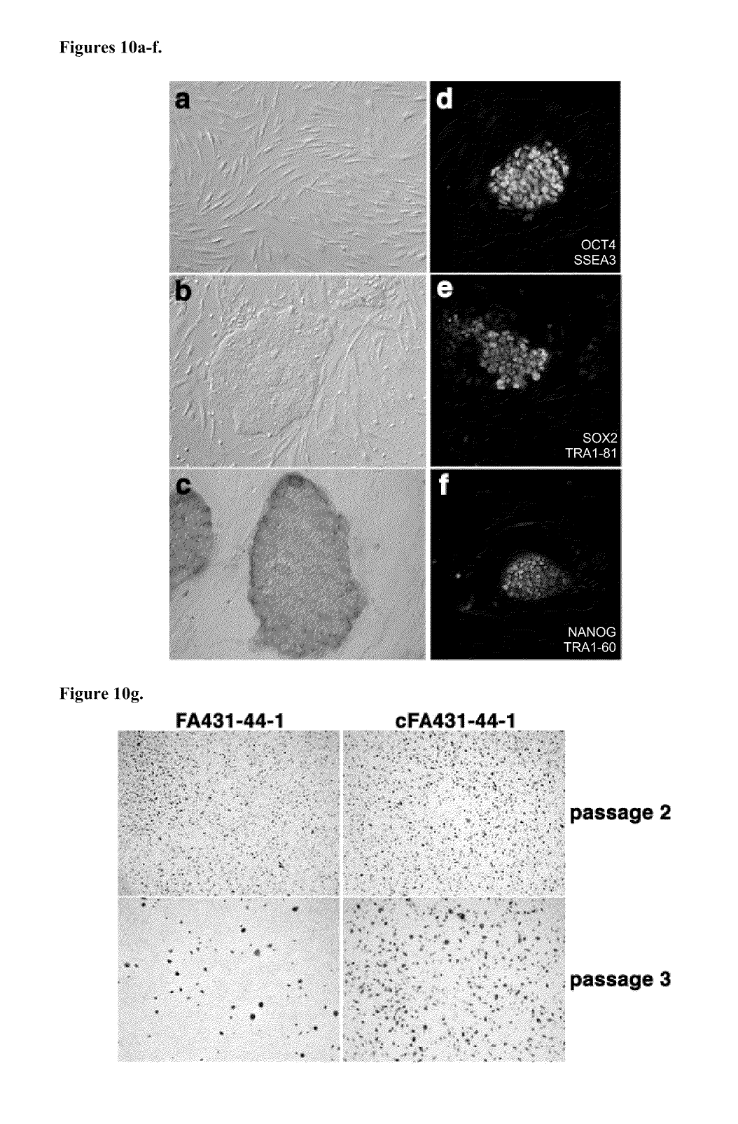

Reprogramming fibroblasts from patient FA431, a FA-D2 patient, was also performed successfully (FIG. 10a). In this case, iPS-like colonies appeared in roughly equal numbers from either unmodified or genetically-corrected fibroblasts (see Table 1). Two iPS-like colonies were picked from either condition, which grew as iPS-like colonies after passaging and stained positive for AP activity (FIGS. 10c, 10g). However, whereas those derived from corrected fibroblasts (cFA431-44-1 and cFA431-44-2) could be maintained in culture for extended periods of time (e.g., at least 18 passages) and showed expression of pluripotency-associated transcription factors and surface markers (FIGS. 10d-10f), those derived from unmodified fibroblasts experienced a progressive growth delay and could not be maintained over the third passage (FIG. 10g). The observation that uncorrected FA-D2 fibroblasts from patient FA431 could be reprogrammed, while iPS cells were only obtained from FANCA-complemented fibroblasts from patients FA90 or FA404, could be explained by the fact that FA-D2 patients, in particular FA431, carry hypomorphic mutations compatible with the expression of residual FANCD2 protein²³.

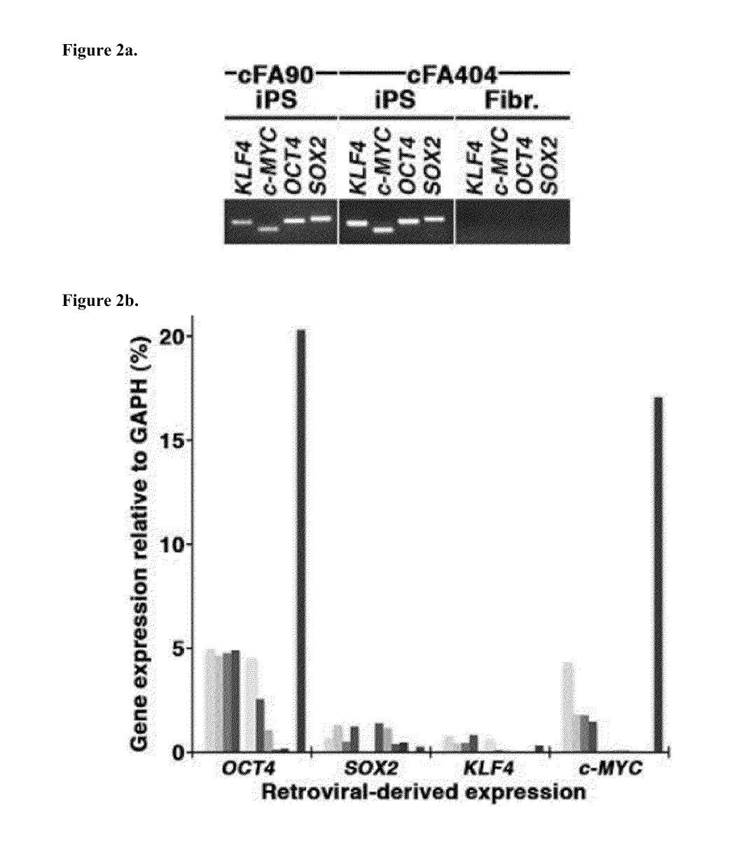

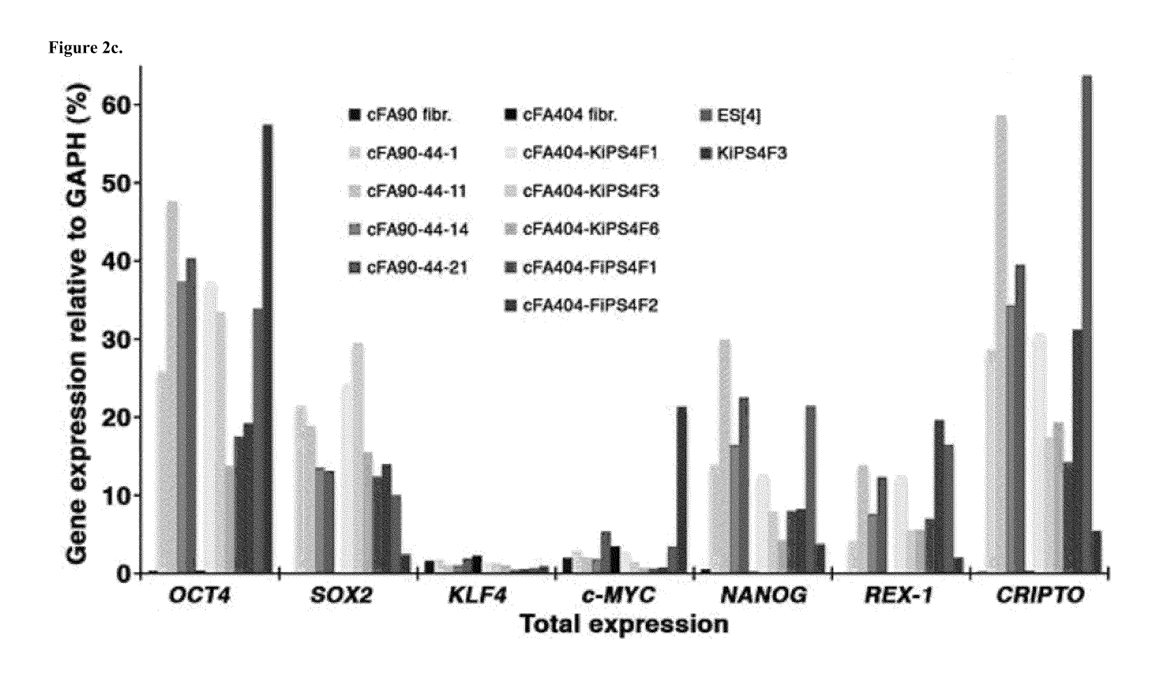

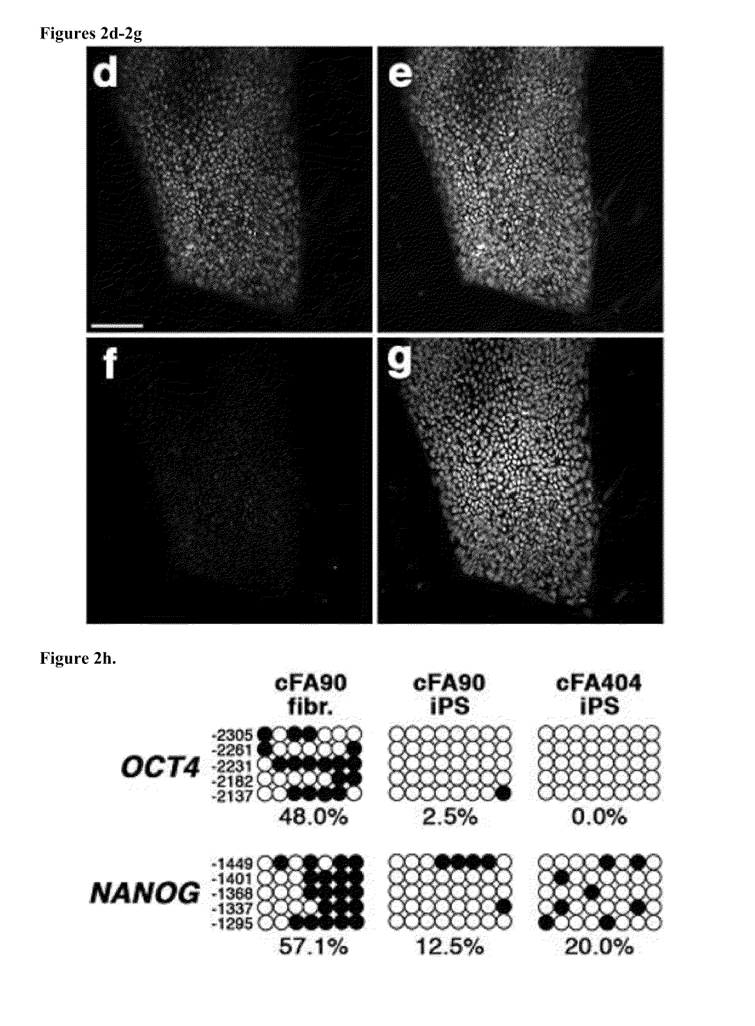

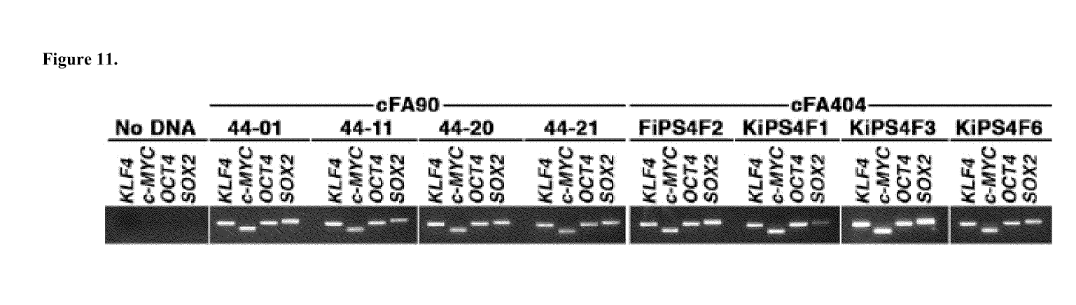

Of the 19 patient-specific iPS cell lines generated in these studies, 10 were selected for a more thorough characterization (see Table 1). The presence of the reprogramming transgenes integrated in their genome was confirmed by PCR of genomic DNA (FIG. 2a and FIG. 11), as well as the origin of the iPS cell lines by comparing their HLA type and DNA fingerprint with those of patients' somatic cells. See Table 2 following. Next, it was analyzed whether the expression of retroviral reprogramming transgenes had been silenced by quantitative RT-PCR analyses using transgene-specific primers. In all lines tested, transgenic expression of the 4 factors was reduced to low or undetectable levels, compared to an iPS cell line (KiPS4F3) that was previously shown not to have silenced the retroviral expression of OCT4 and c-MYC²² (FIG. 2b). Furthermore, all the patient-specific iPS cell lines tested showed re-activation of endogenous OCT4 and SOX2 expression, as well as that of other pluripotency-associated transcription factors such as NANOG, REX-1, and CRIPTO (FIG. 2c). Taking advantage of the fact that the retroviral transgenes used in the studies were FLAG-tagged, it was confirmed, by immunofluorescence, that iPS cells displayed negligible anti-FLAG immunoreactivity (FIGS. 2d-2g). Finally, the promoters of the pluripotency-associated transcription factors OCT4 and NANOG, heavily methylated in patients' fibroblasts, were demethylated in FA-specific iPS cells (FIG. 2h), indicating epigenetic reprogramming to pluripotency.

TABLE 2
Molecular typing of FA fibroblasts and iPS cell lines.

| | cFA90 | | | cFA404 | | | | | |
|---|---|---|---|---|---|---|---|---|---|
| | | iPS cells | | | iPS cells | | | | |
| | Fibr. | 44-11 | 44-14 | Fibr. | FiPS4F1 | FiPS4F2 | KiPS4F1 | KiPS4F3 | KiPS4F6 |
| HLA | | | | | | | | | |
| A* | 02, 29 | 02, 29 | 02, 29 | 02, 24 | 02, 24 | 02, 24 | 02, 24 | 02, 24 | 02, 24 |
| B* | 44, 49 | 44, 49 | 44, 49 | 35, 51 | 35, 51 | 35, 51 | 35, 51 | 35, 51 | 35, 51 |
| Cw* | 07, 16 | 07, 16 | 07, 16 | 02, 04 | 02, 04 | 02, 04 | 02, 04 | 02, 04 | 02, 04 |
| DRB1* | 11, 15 | 11, 15 | 11, 15 | 03, 04 | 03, 04 | 03, 04 | 03, 04 | 03, 04 | 03, 04 |
| DRQ1* | 03, 06 | 03, 06 | 03, 06 | 02, 03 | 02, 03 | 02, 03 | 02, 03 | 02, 03 | 02, 03 |
| DNA | | | | | | | | | |
| fingerprint | | | | | | | | | |
| Amelogenin | X | X | X | X, Y | X, Y | X, Y | X, Y | X, Y | X, Y |
| D3S1358 | 16, 17 | 16, 17 | 16, 17 | 15, 16 | 15, 16 | 15, 16 | 15, 16 | 15, 16 | 15, 16 |
| vWA | 14, 17 | 14, 17 | 14, 17 | 14, 18 | 14, 18 | 14, 18 | 14, 18 | 14, 18 | 14, 18 |
| FGA | 21, 23 | 21, 23 | 21, 23 | 19, 25 | 19, 25 | 19, 25 | 19, 25 | 19, 25 | 19, 25 |
| D8S1179 | 13 | 13 | 13 | 14, 15 | 14, 15 | 14, 15 | 14, 15 | 14, 15 | 14, 15 |
| D21S11 | 28, 30 | 28, 30 | 28, 30 | 27, 29 | 27, 29 | 27, 29 | 27, 29 | 27, 29 | 27, 29 |
| D18S51 | 15 | 15 | 15 | 14, 15 | 14, 15 | 14, 15 | 14, 15 | 14, 15 | 14, 15 |
| D5S818 | 12, 13 | 12, 13 | 12, 13 | 11, 13 | 11, 13 | 11, 13 | 11, 13 | 11, 13 | 11, 13 |
| D13S317 | 11, 12 | 11, 12 | 11, 12 | 12, 13 | 12, 13 | 12, 13 | 12, 13 | 12, 13 | 12, 13 |
| D7S820 | 9, 11 | 9, 11 | 9, 11 | 10, 13 | 10, 13 | 10, 13 | 10, 13 | 10, 13 | 10, 13 |

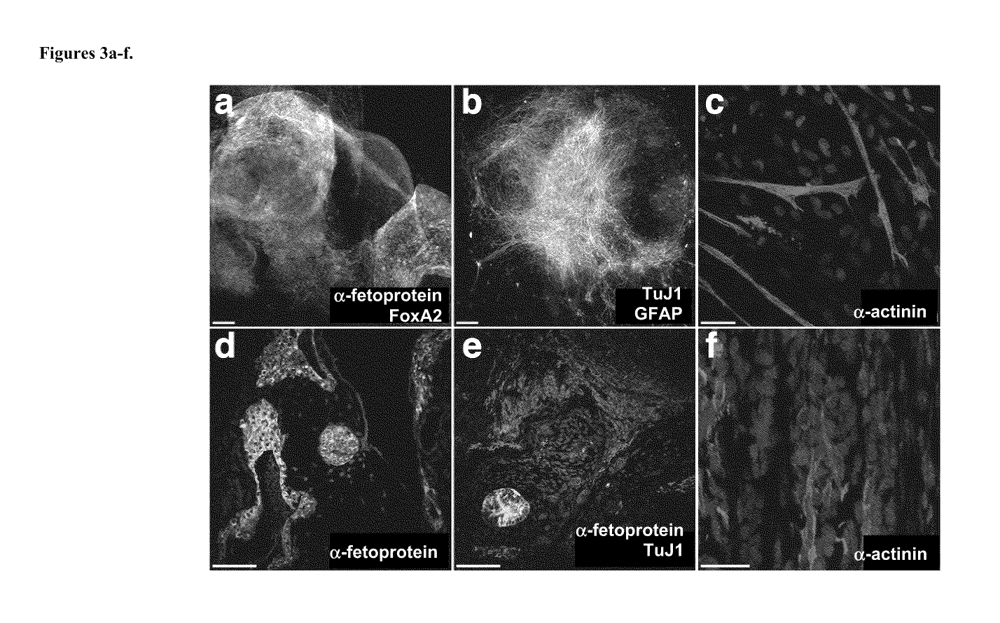

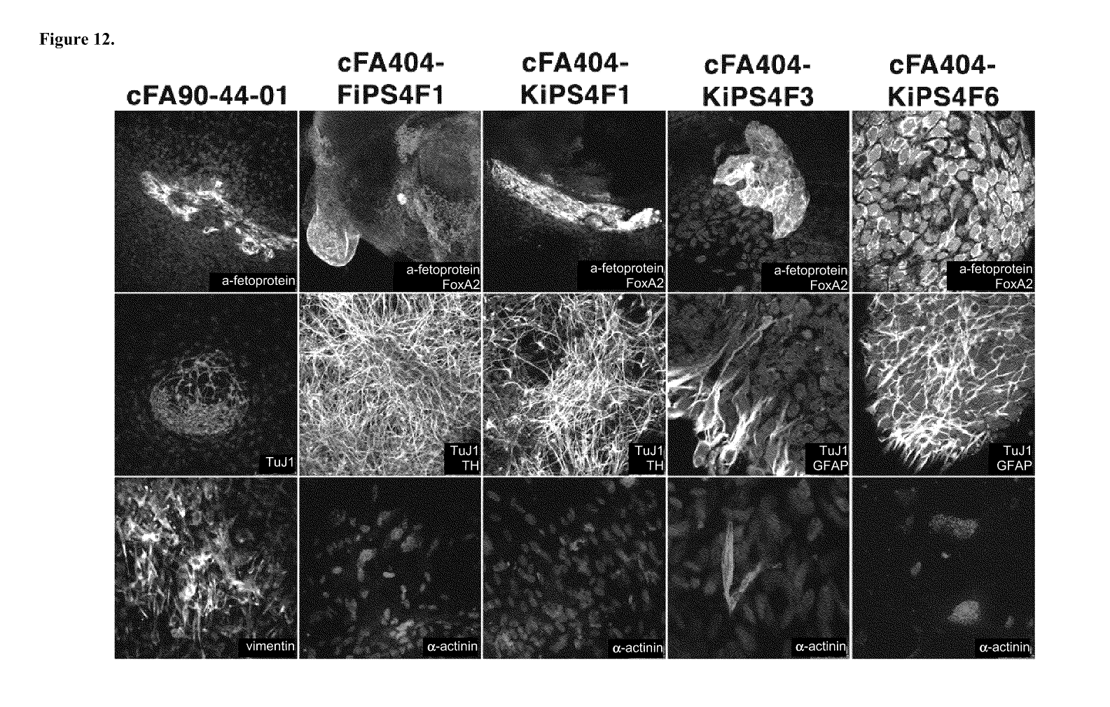

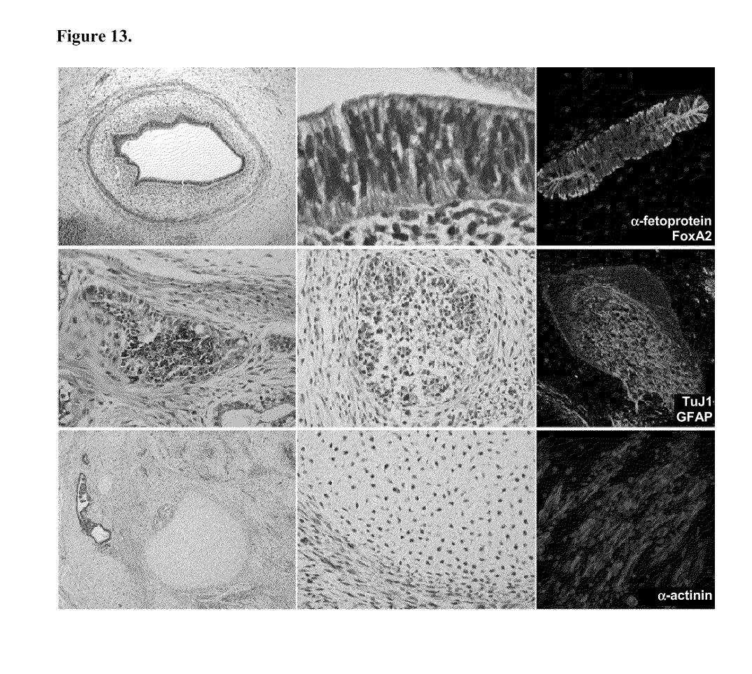

Next the ability of patient-specific iPS cells to differentiate into cell derivatives of all three embryonic germ layers was analyzed. In vitro, iPS-derived embryoid bodies readily differentiated into endoderm, ectoderm and mesoderm derivatives as judged by cell morphology and specific immunostaining with α-fetoprotein/FoxA2, TuJ1/GFAP, and α-actinin, respectively (FIGS. 3a-3c, and FIG. 12). Following specific in vitro differentiation protocols, iPS cells gave rise to specialized mesoderm-derived cell types such as rhythmically beating cardiomyocytes and hematopoietic progenitor cells (see below). The patient-specific iPS cells were also subjected to the most stringent test available to assess pluripotency of human cells, the formation of bona fide teratomas²⁴. For this purpose, cells from 8 different lines were injected into the testes of immunocompromised mice. In all cases, teratomas could be recovered after 8-10 weeks that were composed of complex structures representing the three main embryonic germ layers, including glandular formations that stained positive for definitive endoderm markers, neural structures that expressed neuroectodermal markers, and mesoderm derivatives such as muscle and cartilage (FIGS. 3d-3f, FIG. 13). Using comparable assays, the in vitro differentiation and teratoma induction abilities of a variety of normal human pluripotent stem cell lines, including hES cells²⁵ and iPS cells generated from healthy donors²² has been recently characterized. Overall, no differences were detected in the capacity of FA patient-specific iPS cell lines to differentiate into any of the cell lineages tested, when compared to that of either hES cells or normal iPS cells.

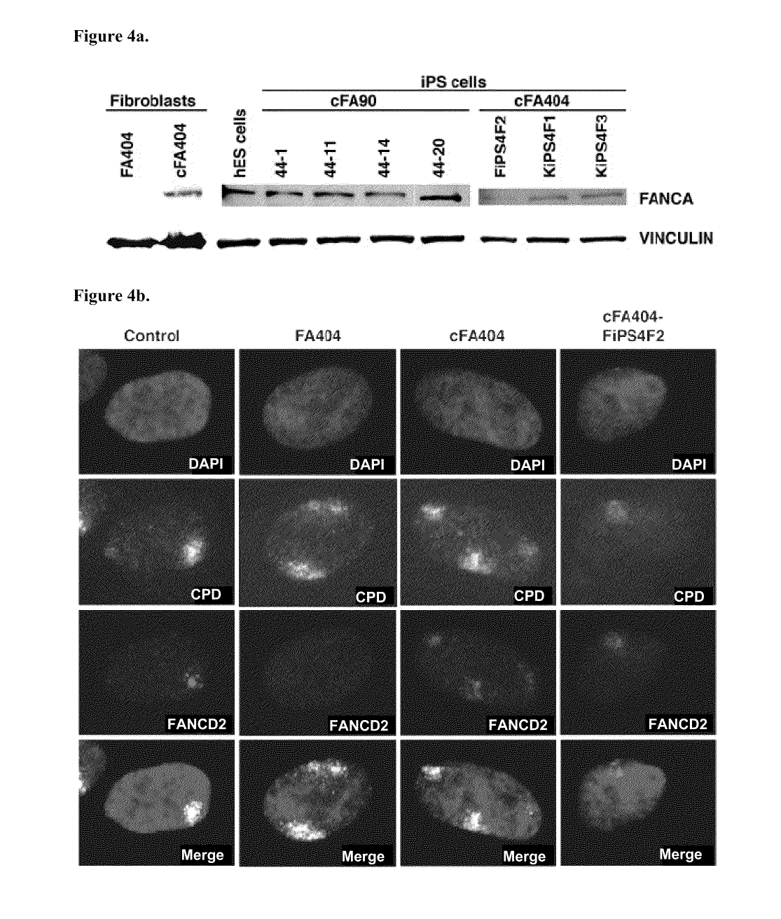

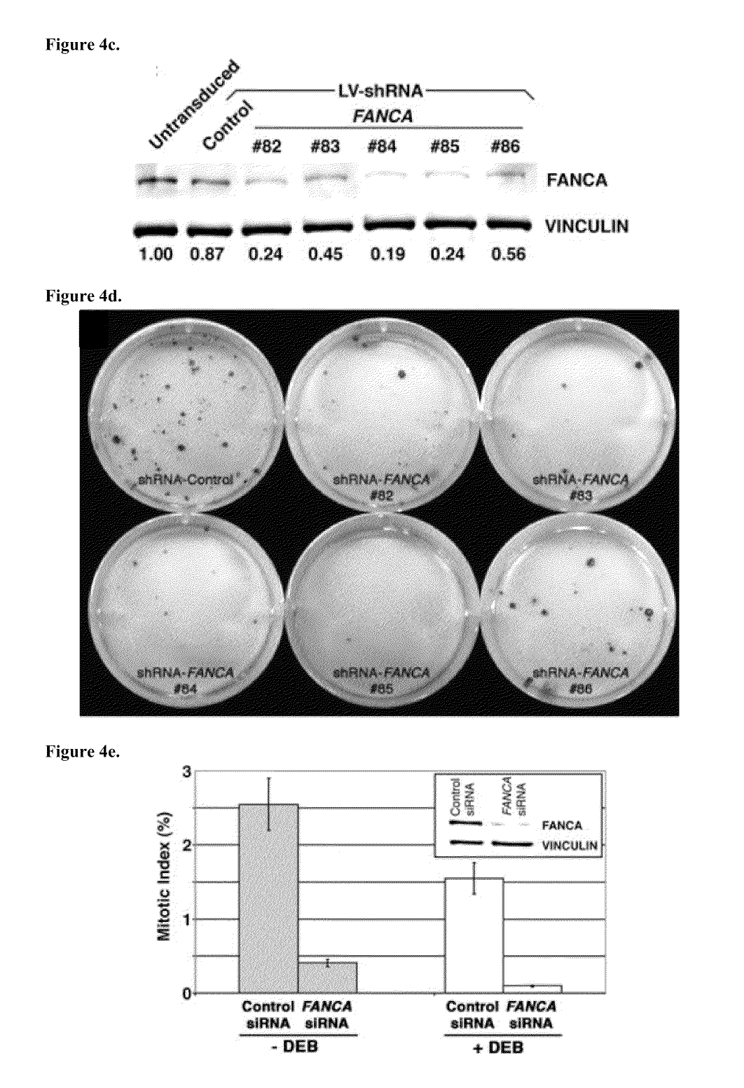

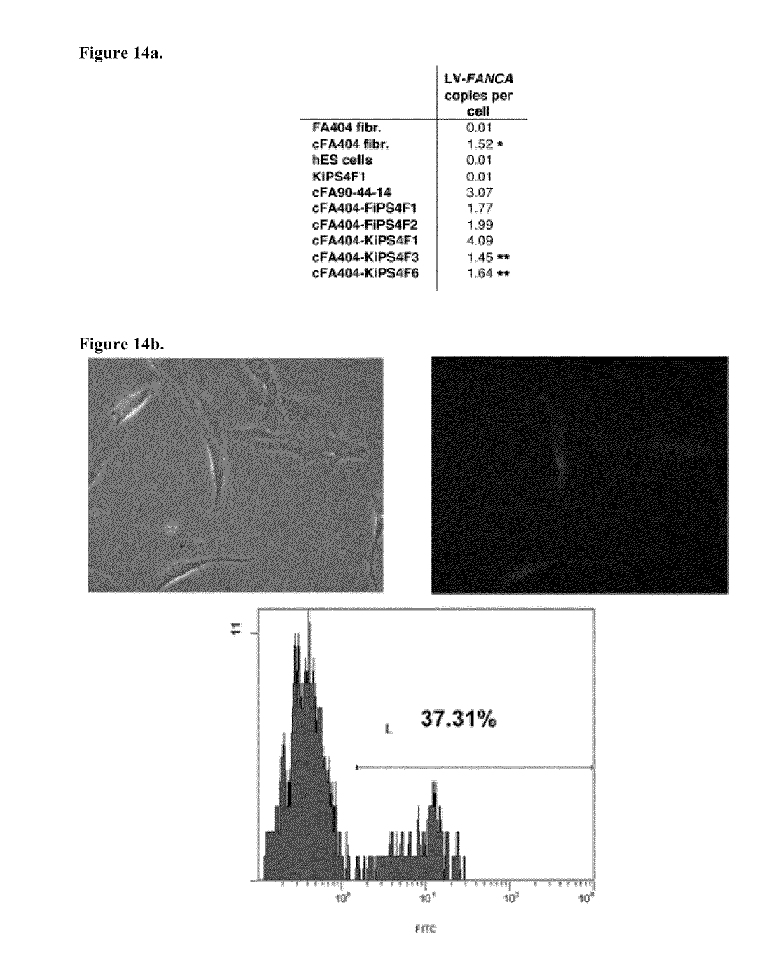

The generation of indefinitely self-renewing iPS cells from patients with monogenic diseases provides a unique opportunity for controlled ex vivo gene therapy. The FA patient-specific iPS cell lines were generated from somatic cells that had been previously transduced with FA-correcting lentiviruses. Indeed, the presence of integrated copies of the gene therapy vectors could be detect by quantitative PCR of genomic DNA in all FA-iPS cell lines tested (FIG. 14a). A concern with gene therapy strategies is the silencing of the correcting transgene. Even though lentiviral transgenes are particularly resistant to silencing in hES cells²⁶, this appears to be promoter-dependent²⁷ and nearly complete silencing has been recently observed in the context of induced reprogramming^{3,8}. In these experiments, a partial degree of silencing of the correcting transgene occurred in FA-iPS cells. The FANCA-expressing lentiviruses used to infect patients' cells carried an EGFP reporter after an IRES element that conferred a weak, but detectable, fluorescence to transduced cells (FIG. 14b). However, EGFP expression was no longer detectable in FA-iPS or FA-iPS-derived cells (data not shown), indicating that the transgene had been, at least, partially silenced. To check the extent of transgene silencing, the expression of FANCA was analyzed, absent in uncorrected fibroblasts from patients FA90 or FA404 (FIG. 4a). All the FA-iPS cell lines analyzed expressed lentiviral-derived FANCA, showing that none of them had completely silenced the transgene (FIG. 4a). While the majority of FA-iPS cell lines expressed FANCA at levels comparable to those of hES cells, the expression in some lines was much lower. In this respect, it has been recently shown that weak expression of FANCA is enough to restore the FA pathway in FA-A cells²¹.

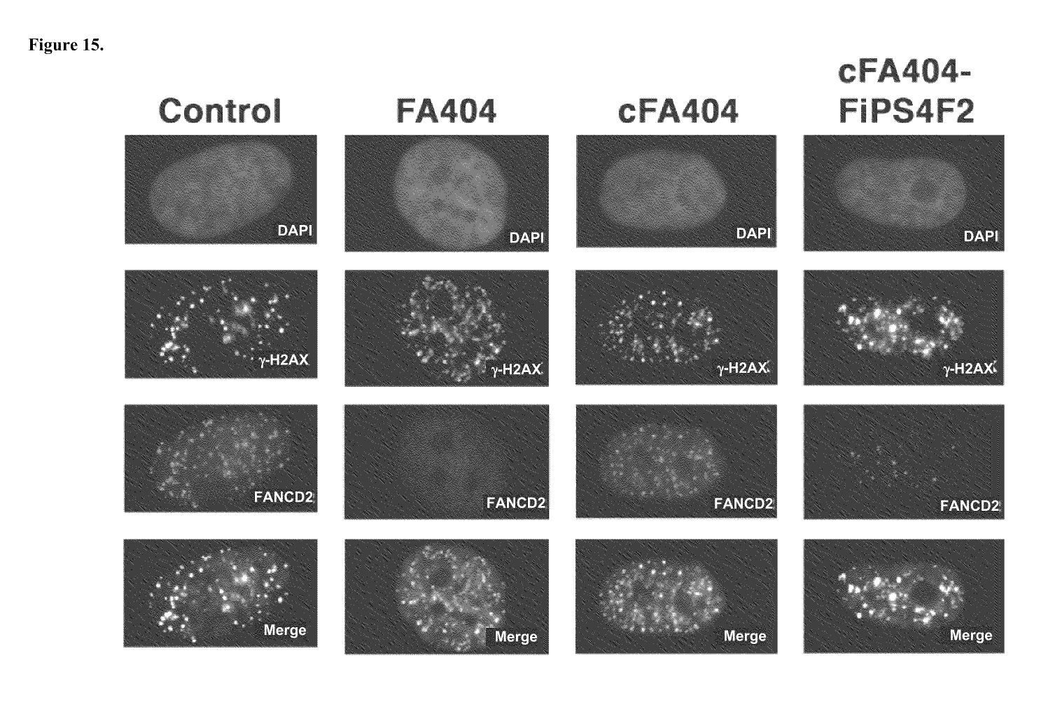

To confirm the disease-free phenotype of FA-iPS cells a battery of functional tests was performed. When the FA pathways is functional, FANCD2 is activated and subsequently relocated to stalled replication forks in a process that depends on FANCA¹⁰. Subnuclear accumulation of stalled replication forks was induced by high-energy local UV-irradiation across a filter with 5 μm pores and checked whether FANCD2 relocated to the locally-damaged subnuclear areas, visualized by immunofluorescence with antibodies against cyclobutane pyrimidine dimers (CPD)²⁸. In those experiments, FANCD2 relocated to stalled replication forks in normal or complemented FA fibroblasts, as well as in fibroblast-like cells derived from FA-iPS cells, but not in uncorrected FA fibroblasts (FIG. 4b). In addition, replication fork collapse was induced by treating FA-iPS-derived cells with the DNA replication inhibitor hydroxyurea (HU). Stalled and broken replication forks were detected by phosphorylated histone H2AX (γ-H2AX) immunoreactivity. Also in this case, it was found that FANCD2 normally relocated to HU-induced stalled replication forks in normal fibroblasts, genetically complemented FA fibroblasts, or FA-iPS-derived cells, but failed to do so in uncorrected FA fibroblasts (FIG. 15). These results, together with the persistent FANCA expression in FA-iPS cells, clearly show that iPS cells generated from genetically corrected FA somatic cells maintain a fully-functional FA pathway and are, thus, phenotypically disease free.

The findings that successful reprogramming of FA cells only occurred in those that had been transduced with FANCA-expressing lentiviruses (in spite of only 35-50% of cells being actually transduced with the correcting lentiviruses; see FIG. 14b), and that lentiviral transgenes were not completely silenced in FA-iPS cells, suggest that a functional FA pathway confers a strong selection advantage for iPS cell generation and/or maintenance. This would be consistent with the marked proliferation advantage observed in hematopoietic stem cells from mosaic FA patients that have spontaneously reverted a pathogenic mutation in one of the affected alleles²⁹⁻³¹ or from FA mice genetically treated with therapeutic lentiviruses³². To directly address this possibility, the transgenic expression of FANCA in FA-iPS cells was knocked down by lentiviral delivery of FANCA-shRNAs. Of the 5 different shRNAs tested, 3 achieved greater than 70% downregulation of FANCA expression in cFA404-KiPS4F3 cells (FIG. 4c). Notably, iPS cells with the lowest FANCA levels failed to proliferate after 1 passage (FIG. 4d). These experiments were repeated with cFA90-44-14 cells and obtained very similar results (data not shown). As a complementary approach, transient downregulation of FANCA expression in FA-iPS-derived cells was induced by siRNA transfection, which led to a marked decrease in cell proliferation (˜7 fold) compared to scramble siRNA-transfected cells (FIG. 4e). The decrease in cell proliferation induced by FANCA siRNA was even more pronounced (>15 fold) in response to diepoxybutane-induced DNA damage (FIG. 4e). These results provide further evidence for the FA disease-free status of the FA patient-specific iPS cells and, importantly, unveil a previously unsuspected role of the FA pathway as a critical player in the maintenance of pluripotent stem cell self-renewal. It is conceivable that the FANCA requirement for normal iPS cell proliferation may have played an important part in ensuring that FA patient-specific iPS cells are maintained disease-free; for instance, by positively selecting iPS cells that have not completely silenced the correcting transgene and express FANCA above threshold levels.

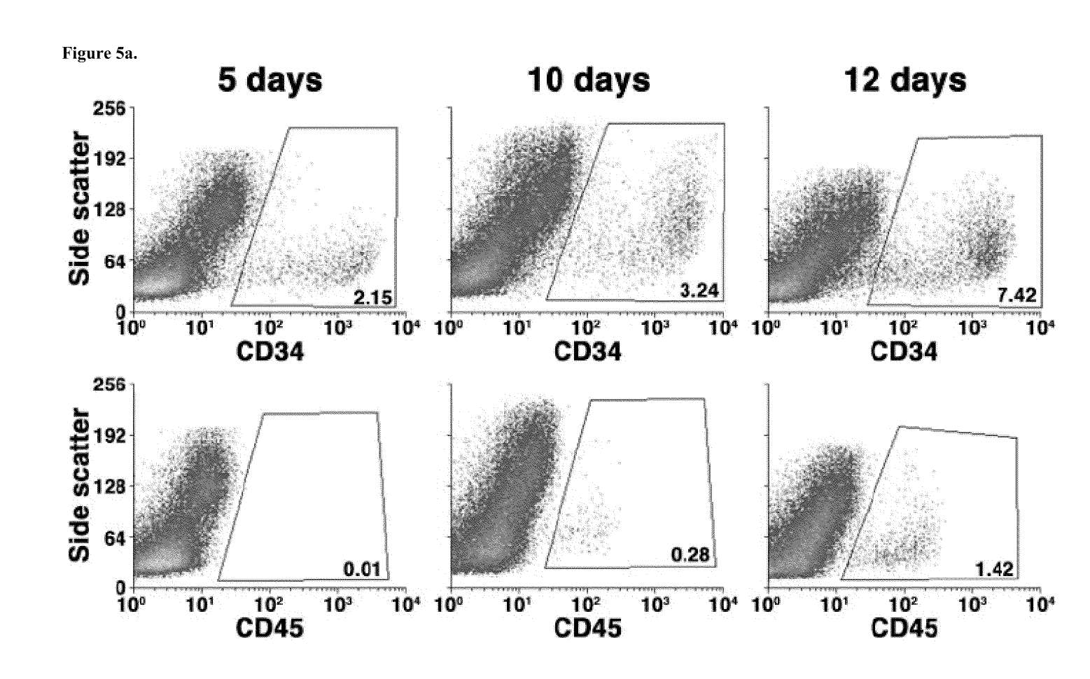

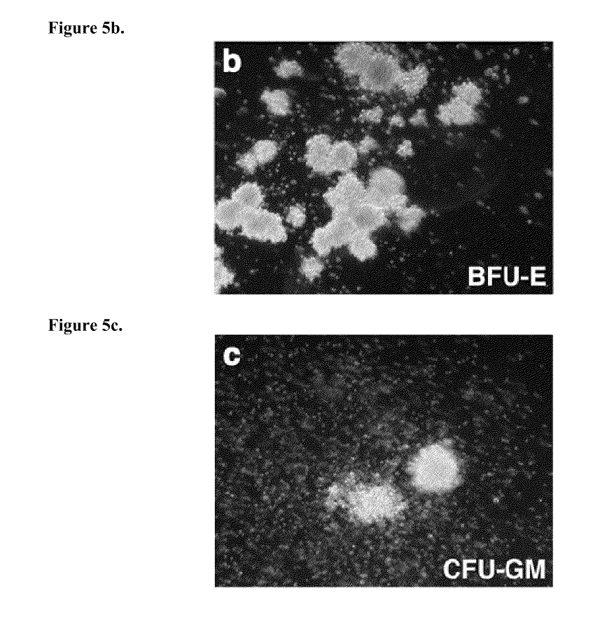

The most prominent feature of FA is BMF arising from the progressive decline in the numbers of functional hematopoietic stem cells¹⁶⁻¹⁸. Therefore, it was tested whether patient-specific iPS cells could be used as a source of hematopoietic cells for potential cell therapy applications. Embryoid bodies from 6 different patient-specific iPS cell lines (cFA90-44-11 and -44-14, cFA404-FiPS4F2, -KiPS4F1, -KiPS4F3, and -KiPS4F6) were used in differentiation experiments based on co-culture with OP9 stromal cells³³ in the presence of hematopoietic cytokines In all cases, CD34⁺ cells could be detected by flow cytometry starting at day 5 and peaking at day 12 (7.23±2.57%, n=7). CD45⁺ cells could also be detected in those cultures from day 10, which reached 0.95±0.38% (n=6) by day 12 (FIG. 5a). The timing of appearance and frequency of hematopoietic progenitors obtained from patient-specific iPS cells were similar to those obtained using iPS cells from healthy individuals (7.24±3.43% of CD34⁺ cells at day 12, n=5 from 2 independent iPS cell lines) and hES cells (6.62±1.03% of CD34⁺ cells at day 12, n=5 from 2 independent hES cell lines; see also ref 34). These results show that FA patient-specific iPS cells display normal potential to undergo early hematopoiesis in vitro.

FA-iPS-derived CD34⁺ cells were purified at day 12 of the differentiation protocol by 2 rounds of magnetic activated cell sorting (MACS) to test their hematopoietic differentiation ability in clonogenic progenitor assays. It could be observed that FA-iPS cell-derived CD34⁺ cells generated erythroid (burst forming unit-erythroid [BFU-E]) and myeloid (colony forming unit-granulocytic, monocytic [CFU-GM]) colonies after 14 days in methylcellulose culture (FIGS. 5b-5c). The myeloid nature of CFU-GM colonies was confirmed by the expression of the CD33 and CD45 markers in these colonies (FIG. 5d). The hematopoietic potential of FA-iPS cell-derived CD34⁺ cells was robust and the numbers of colony-forming cells (CFCs) obtained in clonogenic assays were comparable to those obtained from CD34⁺ cells derived from hES cells or control iPS cells (FIG. 5e, solid bars). These results indicate that patient-specific iPS cells successfully differentiated into hematopoietic progenitors of the erythroid and myeloid lineages. In some experiments, iPS-derived CD34⁺ cells were maintained with hematopoietic growth factors for 7 days. In these cases, the number of CFCs increased very significantly (about 60 fold), suggesting a progressive hematopoietic differentiation in these cultures. It was also attempted to generate blood cells in NOD/SCID mice transplanted with CD34⁺ cells derived from genetically corrected FA-iPS cells, but no engraftments of human hematopoietic cells were observed in those animals, in agreement with previous data showing current technical limitations to repopulate immunodeficient mice with in vitro-differentiated hES cells³⁵.

To test whether hematopoietic progenitors derived from genetically corrected and reprogrammed FA-A cells maintained the disease-free phenotype of FA-iPS cells, hematopoietic colonies were incubated in the presence of mitomycin C, since hypersensitivity to DNA cross-linking agents is a hallmark of FA cells¹¹. The proportion of mitomycin C-resistant colonies obtained from FA-iPS-derived CD34⁺ cells was similar to that obtained from mononuclear bone marrow cells from a healthy donor, or from CD34⁺ cells derived from either hES cells or iPS cells generated from somatic cells of a healthy donor, and contrasted sharply with the hypersensitivity to mitomycin C shown by FA mononuclear bone marrow cells (FIG. 5e, white bars). Moreover, FA-iPS cell-derived CD34+ cells were able to localize FANCD2 to foci of mitomycin C-induced DNA damage (FIG. 5f), demonstrating a functional FA pathway. The maintenance of the disease-free phenotype in FA-iPS-derived hematopoietic progenitors is further supported by the sustained expression of the lentiviral FANCA transgene during the process of in vitro hematopoietic differentiation (data not shown).

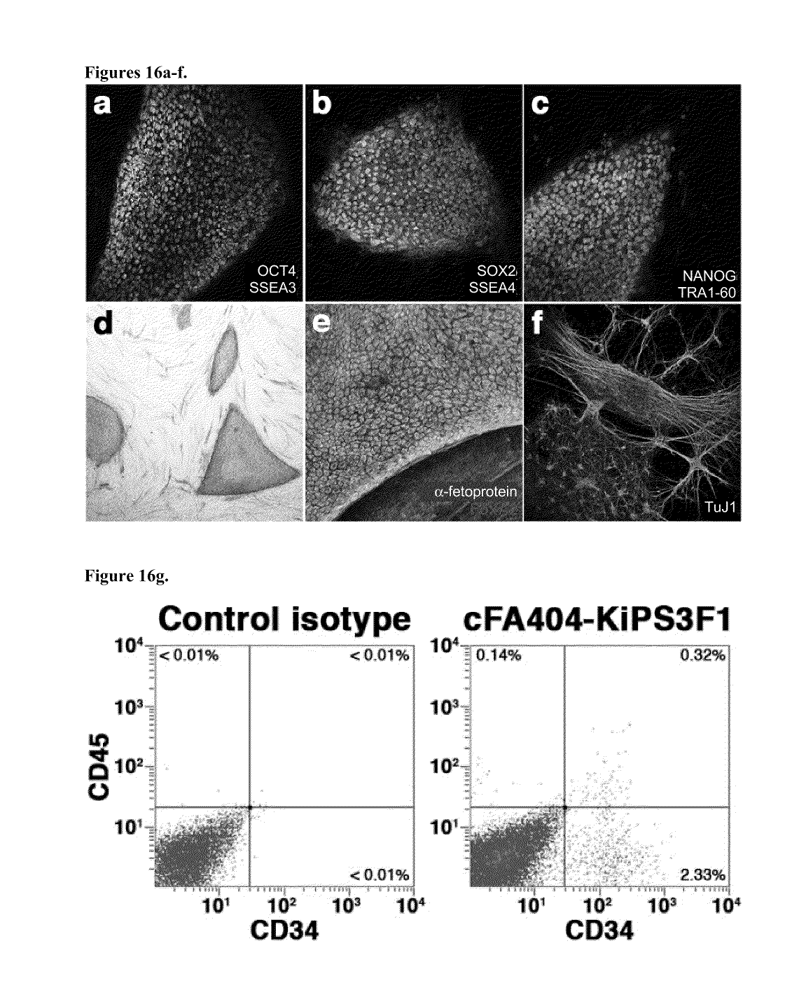

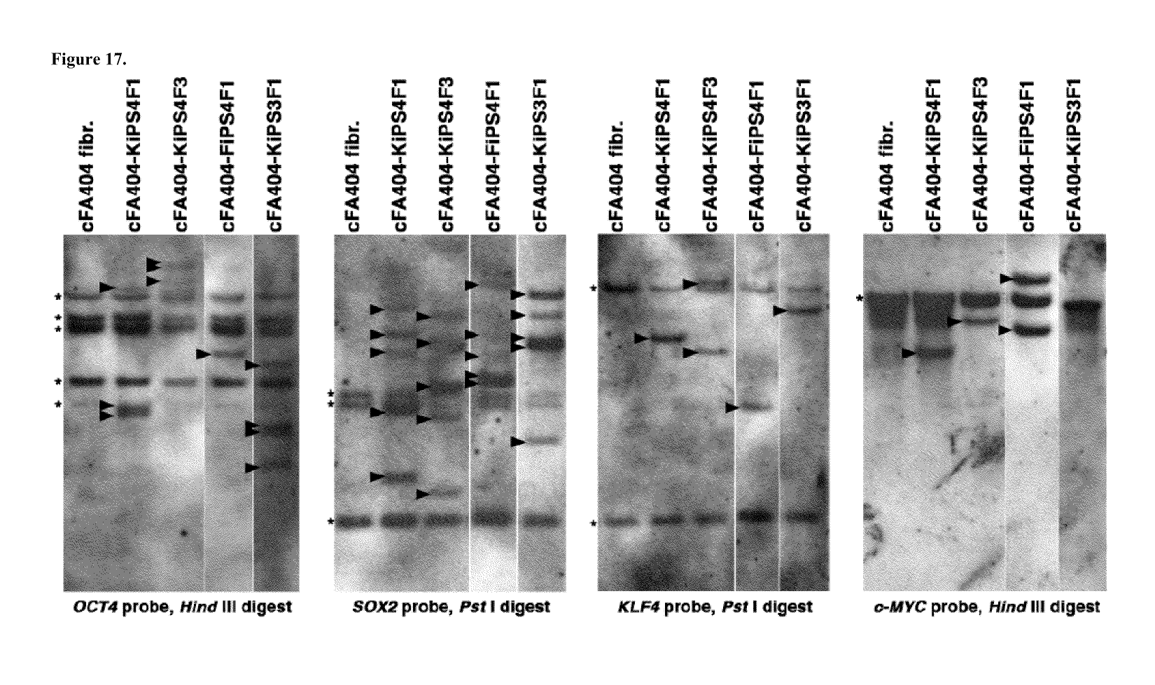

The results demonstrate that iPS cell technology can be used for the generation of patient-specific, disease-corrected cells with potential value for cell therapy applications. Retroviral transduction of adult somatic cells with OCT4, SOX2, KLF4, and c-MYC, while currently the most efficient method for generating human iPS cells, results in permanent undesirable transgene integrations. Although the retroviral transgenes become silenced during reprogramming, their re-activation during cell differentiation (particularly that of the oncogene c-Myc) has been associated with tumor formation³⁶. Human iPS cells can be generated without c-MYC, but reprogramming efficiency in this case is drastically reduced^{22,37}. To ascertain whether FA patient-specific iPS cells could be generated without retroviral transduction with c-MYC, primary keratinocytes from patient FA404 were used. After 3 reprogramming attempts, 1 iPS cell line (cFA404-KiPS3F1) was generated, which expanded robustly and showed all the characteristics and differentiation ability of iPS cells generated with 4 factors, and gave rise to hematopoietic progenitors in vitro (FIG. 16). As expected, the genome of cFA404-KiPS3F1 cells did not contain integrations of the c-MYC retrovirus, as revealed by Southern hybridization with probes specific for the reprogramming factors (FIG. 17) and PCR of genomic DNA (data not shown). The availability of patient-specific iPS cells could overcome the main limitation of current gene therapy strategies, due to risks of insertional oncogenesis⁴⁰, as genetically corrected iPS cells lend themselves to the screening of safe integration sites of the therapeutic transgenes. In addition, the generation of iPS cells from patients with genetic diseases offers the possibility of correcting these cells using gene targeting approaches based on homologous recombination⁴¹. The studies, thus, may represent a step forward in the potential application of iPS technology for regenerative medicine.

### VI. Materials and Methods

### Patients

Studies were approved by the authors' Institutional Review Board and conducted under the Declaration of Helsinki Patients were encoded to protect their confidentiality, and written informed consent obtained. The generation of human iPS cells was done following a protocol approved by the Spanish competent authorities (Comisión de Seguimiento y Control de la Donación de Células y Tejidos Humanos del Instituto de Salud Carlos III). Fanconi anemia patients were diagnosed on the basis of clinical symptoms and chromosome breakage tests of peripheral blood cells using a DNA cross-linker drug. Patients FA5, FA90 and FA153 have been previously described⁴²; patients FA430 and FA431 correspond to patients #2 and #10, respectively, in ref 23. Patient FA404 was subtyped by analyzing the G2-phase arrest of dermal fibroblasts transduced with EGFP and FANCA retroviral vectors and then exposed to mitomycin C, as previously described⁴².

### Cell Lines

293T and HT1080 cells (ATCC CRL-12103) were used for the production and titration of lentiviruses, respectively. These cell lines were grown in Dulbecco's modified medium (DMEM, Invitrogen) supplemented with 10% fetal bovine serum (FBS; Biowhitaker™). The ES[2] and ES[4] lines of hES cells were maintained as originally described²⁵. The control iPS cell lines KiPS4F1 and KiPS3F1 and the partially-silenced KiPS4F3 cell line were cultured as reported²².

### Generation of iPS Cells

Fibroblasts were cultured in DMEM supplemented with 10% FBS (all from Invitrogen) at 37° C., 5% CO₂, 5% O₂ and used between 2-6 passages. For reprogramming experiments, about 50,000 fibroblasts were seeded per well of a 6-well plate and infected with a 1:1:1:1 mix of retroviral supernatants of FLAG-tagged OCT4, SOX2, KLF4, and c-MYC^{T58A} (ref 22) in the presence of 1 μg/ml polybrene. Infection consisted of a 45-min spinfection at 750×g after which supernatants were left in contact with the cells for 24 h at 37° C., 5% CO₂. One or two rounds of 3 infections on consecutive days were performed at the times indicated in Supplementary Text. Five days after beginning the last round of infection, fibroblasts were trypsinized and seeded onto feeder layers of irradiated human foreskin fibroblasts in the same culture medium. After 24 h, the medium was changed to hES cell medium, consisting on KO-DMEM (Invitrogen) supplemented with 10% KO-Serum Replacement (Invitrogen), 0.5% human albumin (Grifols, Barcelona, Spain), 2 mM Glutamax (Invitrogen), 50 μM 2-mercaptoethanol (Invitrogen), non-essential amino acids (Cambrex), and 10 ng/ml bFGF (Peprotech). Cultures were maintained at 37° C., 5% CO₂, with media changes every other day. Starting 1 week after plating onto feeders, medium was supplemented with 1 μM PD0325901 and 1 μM CT99021 (both from Stem Cell Sciences) for 1 week. Colonies were picked based on morphology 45-60 d after the initial infection and plated onto fresh feeders. Lines of patient-specific iPS cells were maintained by mechanical dissociation of colonies and splitting 1:3 onto feeder cells in hES cell medium or by limited trypsin digestion and passaging onto Matrigel-coated plates with hES cell medium pre-conditioned by mouse embryonic fibroblasts (MEFs). Other inhibitors were used as indicated in Supplementary Text, at the following concentrations: 10 μM U0126 (Calbiochem), 25 μM PD098059 (Calbiochem), 5 μM BIO (Sigma), 10 μM Y27632 (Calbiochem). Generation of patient-specific KiPS cells was essentially as previously reported²², except that primary epidermal keratinocytes were derived from small biopsy explants in the presence of irradiated fibroblasts in DMEM/Hams-F12 (3:1) supplemented with 10% FBS, 1 μg/mlEGF (BioNova), 0.4 μg/ml hydrocortisone, 5 μg/mlTransferrin, 5 μg/ml Insulin, 2×10⁻¹¹ M Liothyronine (all from Sigma), and 10⁻¹⁰ M cholera toxin (Quimigen).

### Quantitative RT-PCR, Transgene Integration, and Promoter Methylation Analyses

Expression of retroviral transgenes and endogenous pluripotency-associated transcription factors, integration of retroviral transgenes by genomic PCR or Southern blot, and methylation status of OCT4 and NANOG promoters were assessed as previously reported²².

### HLA Typing and DNA Fingerprinting

Molecular typing of cell lines was performed by Banc de Sang i Teixits (Barcelona, Spain). HLA typing hES cell lines used sequence-based typification (SBT) with the AlleleSEQR® HLA Sequencing Kit (Atria Genetics). Microsatellite DNA fingerprinting was performed using multiplex polymerase chain reaction of 9 microsatellites/short tandem repeats (STRs) plus Amelogenin gene using AmplF1STR® Profiler Plus Kit (Applied Biosystems).

### Analysis of Proviral Copy Number and Transgene Expression

Quantification of proviral copy number per cell was analyzed by qPCR in a Rotor Gene™ RG-3000 (Corbett Research Products) using primers against FANCA transgene: hFANCA-F: 5′-GCTCAAGGGTCAGGGCAAC-3′ (SEQ ID NO:9) and hFANCA-R: 5′-TGTGAGAAGCTCTTTTTCGGG-3′ (SEQ ID NO:10) and detected with the Taqman® probe FANCA-P: 5′-FAM-CGTCTTTTTCTGCTGCAGTTAATACCTCGGT-BHQ1-3′ (SEQ ID NO:11). To quantify the number of cells, actin primers were used: DNA-RNA-β Actin-F: 5′-ATTGGCAATGAGCGGTTC C-3′ (SEQ ID NO:12) and DNA-β Actin-R: 5″-ACAGTCTCCACTCACCCAGGA-3′ (SEQ ID NO:13) and detected with the probe DNA-RNA-β Actin-P: 5′-Texas Red-CCCTGAGGCACTCTTCCAGCCTTCC-BHQ1-3′ (SEQ ID NO:14). To measure the average proviral DNA per transduced cell a standard curve of LV: (FANCA-IRES-EGFP) and β Actin DNA amplification was made. Next, the average proviral number per cell was estimated by interpolation of the hFANCA β Actin ratio from each DNA sample in the standard curve. The expression of the human FANCA transgene was analyzed by real-time quantitative reverse transcriptase-polymerase chain reaction (qRT-PCR) on cDNA obtained from total RNA. Samples from a healthy donor and a FA patient were used as controls. To distinguish between endogenous expression of hFANCA and the expression due to the transgene, total hFANCA expression was analyzed using hFANCA primers and probe and the endogenous expression was analyzed using h3′FANCA-F: TCTTCTGACGGGACCTGCC (SEQ ID NO:15) and h3′FANCA-R: AAGAGCTCCATGTTATGCTTGTAATAAAT (SEQ ID NO:16) and detected with Taqman® probe: h3′FANCA-P: 5′-FAM-CACACCAGCCCAGCTCCCGTGTAA-BHQ1-3′ (SEQ ID NO:17). For housekeeping control expression β Actin was analyzed using DNA-RNA-β Actin-F primer, RNA-β Actin-R primer: 5″-CACAGGACTCCATGCCCA-3′ (SEQ ID NO:18) and Taqman® probe DNA-RNA-β Actin-P. Differences between the expression obtained with the hFANCA and the h3′FANCA indicate the expression of the integrated provirus.

### Western Blot

Cell extracts were prepared using standard RIPA buffer. Briefly, harvested cells were washed three times with PBS and then resuspended in RIPA buffer. The total protein concentration in the supernatant was then measured using the Bio-Rad Protein Assay (Biorad, Hercules, Calif., USA) according to the manufacturer's instructions. 40 μg of total proteins were then loaded on a 6% SDS-PAGE and subjected to standard Western blot procedure followed with immunodetection with an anti-human FANCA antibody kindly provided by the Fanconi Anemia Research Fund, Eugene, Portland, USA. Vinculin (Abcam, Cat. No. ab18058; 1:5000) was used as internal loading control.

Functional Studies of the FA Pathway in iPS-Derived Cells

Subnuclear accumulation of stalled replication forks was induced by local UVC irradiation essentially as described²⁸ with some minor modifications. Briefly, cells (primary fibroblasts or iPS-derived cells) were seeded on 22×22 mm sterile coverslips. Prior to irradiation, the medium was aspirated and the cells were washed with PBS. Cells were then covered with an Isopore™ polycarbonate filter with pores of 5 μm diameter (Millipore, Badford, Mass., USA) and exposed to 60 J/m² UVC from above with a Philips 15 W UV-C lamp G15-T8. Subsequently, the filter was removed and fresh pre-warmed medium added back to the cells, which were returned to culture conditions and processed for immunofluorescence 6 h later. In parallel experiments, primary fibroblast and iPS-derived cells were exposed to hydroxyurea (HU, 2 mM) for 24 h and then fixed and processed for immunofluorescence as described below.

For FANCD2 detection at UV-induced stalled replication forks, cells were fixed with PBS containing 4% formaldehyde (Sigma-Aldrich, St. Louis, Mo., USA) for 15 min at room temperature (RT), washed with PBS and incubated with PBS, 0.5% Triton (Sigma-Aldrich) for 10 min at RT. Next, cells were washed with PBS and subsequently rinsed with a washing buffer (WB) consisting of 5% bovine albumin (Sigma-Aldrich) and 0.05% Tween-20 (Sigma-Aldrich) in PBS. Cells were then treated with 1M HCl for 5 min at 37° C. and incubated for 1 h at 37° C. with a primary rabbit antibody against FANCD2 (Abcam, Cambridge, UK; 1:1000) mixed with an anti CPD antibody (Kamiya Biomed, MC-062; 1:500). Cells were then washed for 15 min in WB with gentle agitation and incubated with secondary antibodies anti-mouse Alexa Fluor® 488 (Molecular Probes, Eugene, Oreg., USA) and anti-rabbit Alexa Fluor® 555 (Molecular Probes) diluted in WB for 30 min at 37° C. followed by a 15 min washing step in WB with gentle agitation, rinsed in distilled water, air dried and mounted in anti-fading medium containing 4′-6′-diamidino-2-phenylindole (DAPI, Sigma). In the HU experiments, immunodetection was identical with the exception that the HCl washing step and a primary mouse antibody against anti-γH2AX (Upstate; 1:3000) was used instead of an anti-CPD to visualize nuclei foci representing stalled and broken replication forks. Using this color combination, nuclei were visualized in blue color, the site of stalled replication forks (UV irradiation spot or HU-induced foci) in green color, and FANCD2 in red color. Microscopic analysis and image capturing were performed in identical optical and exposure conditions for all cell types using a Zeiss Axio Observer Al epifluorescence microscope equipped with a AxioCam MRc 5 camera and the AxioVision™, Rel. 4.6 software.

### Immunofluorescence and AP Analyses

Patient-specific iPS cells were grown on plastic coverslide chambers and fixed with 4% paraformaldehyde (PFA). The following antibodies were used: Tra-1-60 (MAB4360, 1:100), Tra-1-81 (MAB4381, 1:100), and Sox2 (AB5603, 1:500) from Chemicon, SSEA-4 (MC-8,3-70, 1:2) and SSEA-3 (MC-631, 1:2) from the Developmental Studies Hybridoma Bank at the University of Iowa, Tujl (1:500; Covance), TH (1:1000; Sigma), α-fetoprotein (1:400; Dako), α-actinin (1:100; Sigma), Oct-3/4 (C-10, SantaCruz, 1:100), Nanog (Everest Biotech; 1:100), GFAP (1:1000; Dako), Vimentin (1:500, Chemicon), FoxA2 (1:100; R&D Biosystems). Secondary antibodies used were all the Alexa Fluor® Series from Invitrogen (all 1:500). Images were taken using Leica SP5 confocal microscope. Direct AP activity was analyzed using an Alkaline Phosphatase Blue/Red Membrane Substrate solution kit (Sigma) according to the manufacturer guidelines. For FANCD2 immunofluorescence assays, cells were grown in plastic coverslide chambers and treated with 30 nM mitomycin C. After 16 h cells were fixed with 3.7% PFA in PBS for 15 minutes followed by permeabilization with 0.5% Triton X-100 in PBS for 5 min. After blocking for 30 minutes in blocking buffer (10% FBS, 0.1% NP-40 in PBS), cells were incubated with polyclonal rabbit anti-FANCD2 antibody (Novus Biologicals, NB 100-182, 1/250). Anti-rabbit Texas red conjugated antibody (Jackson Immunoresearch Laboratories) was used as secondary antibody (1:500). Slides were analyzed with a fluorescence microscope Axioplan2 (Carl Zeiss, Gottingen, Germany) using a 100×/1.45 Oil working distance 0.17 mm objective.

### In Vitro Differentiation

Differentiation towards endoderm, cardiogenic mesoderm, and neuroectoderm was carried out essentially as described²⁵. Differentiation towards fibroblast-like cells was accomplished by plating embryoid bodies (EBs) onto gelatin-coated plates in 90% DMEM, 10% FBS and repeated passaging of differentiated cells with fibroblast-like morphology. For hematopoietic differentiation, EBs were produced by scraping of confluent iPS wells and cultured in suspension in EB medium (90% DMEM, 10% FBS) for 24-48 hrs. EBs were then placed over a feeder layer of confluent OP9 stromal cells and allowed to attach. The medium used for the first 48 h of differentiation was 50% EB medium and 50% hematopoietic differentiation medium. The hematopoietic differentiation medium was StemSpan® Serum Free Medium (StemCell Technologies) supplemented with cytokines BMP4 (10 ng/ml), VEGF (10 ng/ml), SCF (25 ng/ml), FGF (10 ng/ml), TPO (20 ng/ml), and Flt ligand (10 ng/ml). After 48 h, cells were cultured with hematopoietic differentiation medium, with medium changes every 48 h until the end of the differentiation protocol, day 13 after EB plating. At day 13, OP9 and EBs were collected by trypsinization (0.25% trypsin), washed and labeled with anti CD34-beads conjugated antibody (Miltenyi Biotec) according to manufacturer's specification. The CD34⁺ fraction was purified by MACS, and fraction purity was increased by a second round of MACS. Final purity of the collected cells for CD34 was checked on a fraction of the MACS eluate by flow cytometry. The remaining CD34⁺ cells were frozen in medium IMDM containing 10% DMSO and 20% FBS and stored in liquid nitrogen until further use. For the assessment of colony forming cells (CFCs), samples were cultured in triplicates, in Methocult® H4434 (Stem Cell Technologies) at 37° C., in 5% CO₂, 5% O₂ and 95% humidified air. Colonies were scored after two weeks in culture. To analyze the mitomycin C-resistance of the hematopoietic progenitors, CFC cultures were treated with 10 nM mitomycin C (Sigma). In some experiments, iPS-derived CD34⁺ cells were cultured for 7 days in StemSpan® Serum Free Medium (StemCell Technologies) supplemented with hematopoietic growth factors SCF (Amgen, 300 ng/ml), TPO(R&D Systems, 100 ng/ml), and Flt ligand (BioSource, 100 ng/ml).

### Flow Cytometry Analyses

For surface phenotyping the following fluorochrome (phycoerythrin [PE], or allophycocyanin [APC])—labeled monoclonal antibodies were used (all from Becton Dickinson Biosciences): anti-CD34 PE (581/CD34), anti-CD45 APC (HI30). Gating was done with matched isotype control mAbs. Hoechst 33528 (H258) was included at 1 μg/mL in the final wash to exclude dead cells. All analyses were performed on a MoFlo™ cell sorter (DakoCytomation) running Summit software. To analyze the phenotype of hematopoietic progenitors, CFU-GM colonies were picked and washed with PBS. Cells were stained with antihuman CD45-PECy5 mAb (Clone J33, Immunotech) in combination with antihuman CD33-PE mAb (D3HL60.251, Immunotech). Cells were then washed in PBA (phosphate-buffered salt solution with 0.1% BSA and 0.01% sodium azide), resuspended in PBA+2 μg/mL propidium iodide, and analyzed using an EPICS ELITE-ESP cytometer (Coulter). Off-line analysis was done with CXP Analysis 2.1 flow-cytometry software (Beckman Coulter Inc).

### Teratoma Formation

Severe combined immunodeficient (SCID) beige mice (Charles River Laboratories) were used to test the teratoma induction capacity of patient-specific iPS cells essentially as described²². All animal experiments were conducted following experimental protocols previously approved by the Institutional Ethics Committee on Experimental Animals, in full compliance with Spanish and European laws and regulations.

Genetic Correction of FA Cells with Lentiviral Vectors

Lentiviral (LV) vectors carrying the hFANCA-IRES-EGFP cassette under the control of the internal spleen focus forming virus (SFFV) U3 promoter (FANCA-LV; ref. 21) were used to transduce fibroblasts and keratinocytes from FA-A patients. Fibroblasts from the FA-D2 patient were transduced with a LV carrying the FANCD2 cDNA under the control of the vav promoter (FANCD2-LV, ref 21). Lentiviral vectors carrying either of these promoters were equally efficient to correct the phenotype of human FA cells²¹. Vector stocks of VSV-G pseudotyped LVs were prepared by four-plasmid calcium phosphate-mediated transfection in 293T cells, essentially as described⁴³. Supernatants were recovered 24 h and 48 h after transfection and filtered through 0.45 μm. Functional titers of infective LVs were determined in HT1080 cells, plated at 3.5×10⁴ cells per well in 24 well-plates and infected overnight with different dilutions of either LV-supernatant. Cells were washed and incubated with fresh medium, and the proportion of EGFP+ cells was determined 5 days later by flow cytometry, or after 8 days by qPCR.

### Knockdown of FANCA

Lentiviral vectors expressing scramble shRNA and 5 different FANCA-shRNAs (Sigma, MISSION shRNA NCBI accession gi:NM_{—}000135) were used to generate viral particles according to the manufacturer's instructions. For infection, FA patient-specific iPS cells were incubated with viral supernatants in 6-well plates for 24 hours. Puromycin selection (2 μg/ml) was applied for 24 hours 3 days after lentiviral infection and cells were allowed to recover for 3 days before splitting. Transient RNA interference experiments with siRNA were performed as previously described⁴⁴. In brief, cells were grown in OPTI-MEM® medium (Gibco, Cat. No. 31985) with 10% FCS without antibiotics and transfected with 10 nM FANCA siRNA (ref. 45) or Luciferase siRNA as a control (5′CGUACGCGGAAUACUUCGA[dT][dT]3′) (SEQ ID NO:19), with Lipofectamin™ RNAiMAX transfection reagent (Invitrogen, Cat. No. 13778-075) twice over a period of 24 h. 24 h after the second transfection, cells were left untreated or were treated with diepoxybutane (DEB) at 0.02 μg/ml for 3 days and subsequently harvested for protein lysates or processed following standard cytogenetic methods. Mitotic indexes were calculated by counting the number of mitotic cells in 500-6000 cells per point in duplicate. The Luciferase siRNA (SEQ ID NO:19) is a combined DNA/RNA molecule having deoxythymidine at positions 20-21.

### Optimization of the Fibroblast Reprogramming Protocol

Primary dermal fibroblasts from a foreskin biopsy of a healthy donor (HD) were first used to optimize the reprogramming protocol. For this purpose, about 50,000 fibroblasts were transduced at days 0, 1, and 2 with murine stem cell virus-(MSCV) based retroviruses encoding N-terminal FLAG-tagged versions of OCT4, SOX2, KLF4 and c-MYC. Transduced HD fibroblasts were passaged on day 5 onto a feeder layer of mitotically-inactivated primary human fibroblasts and switched to human embryonic stem (hES) cell medium on day 6. Under these conditions, hundreds of “granulated” colonies' appeared starting around day 13 and 3-4 iPS-like colonies were apparent at day 30 (data not shown, see also ref 47). However, iPS-like colonies from Fanconi anemia (FA) fibroblasts using this protocol could not be obtained, even though “granulated” colonies, albeit at reduced numbers, appeared at comparable times. Next it was attempted to increase the efficiency of fibroblast reprogramming by experimental manipulations reported to improve ES cell derivation and/or maintenance, such as inhibition of MEK/ERK signaling^{48,49}, glycogen synthase kinase-3 (GSK3) activity^{50,51} or Rho-associated kinase (ROCK) activity⁵². Treatment with the MEK inhibitors U0126 or PD098059, the GSK3 inhibitor BIO, or the ROCK inhibitor Y27632 during days 6-20 or 13-20 did not increase the numbers of granulated or iPSlike colonies obtained from HD fibroblasts (data not shown). In contrast, combined inhibition of MEK1 and GSK3 with inhibitors PD0325901 and CT99021 (a combination termed 21 that enhances derivation and growth of mouse ES cells⁵³) during days 13-20 of the reprogramming protocol resulted in few small “granulated” colonies that disappeared over the following week, whereas ˜20-30 compact and well defined colonies appeared starting around day 20 (FIGS. 6a, 6b). These colonies could be readily expanded and grew as small, round compact cell colonies, highly reminiscent of mouse ES cells, although they did not express detectable levels of endogenous pluripotency-associated transcription factors or surface markers, nor were they able to undergo in vitro differentiation or to induce teratoma formation upon injection into immunocompromised mice (FIGS. 6c, 6d). Analysis of retroviral integration by PCR on genomic DNA only detected the presence of OCT4- and/or c-MYC-encoding retroviruses in those cell lines (FIG. 6e), indicating that fibroblasts had been immortalized, rather than reprogrammed, but that combined MEK1/GSK3 inhibition efficiently selected cells that had acquired self-renewing ability.

Based on these results, the reprogramming protocol was modified as to include a second round of retroviral infection with the four factors at days 5-7, while maintaining the 21-selection step at days 17-24. Under these conditions, HD fibroblasts reprogrammed to pluripotency and dozens of iPS-like colonies appeared from day 30 to 60 (42±17 AP+colonies of hES-like morphology, n=3).

### VII. References

1. Takahashi, K. & Yamanaka, S. Induction of pluripotent stem cells from mouse embryonic and adult fibroblast cultures by defined factors. Cell 126, 663-676 (2006).
2. Takahashi, K. et al. Induction of pluripotent stem cells from adult human fibroblasts by defined factors. Cell 131, 861-872 (2007).
3. Yu, J. et al. Induced pluripotent stem cell lines derived from human somatic cells. Science 318, 1917-1920 (2007).
4. Park, I. H. et al. Reprogramming of human somatic cells to pluripotency with defined factors. Nature 451, 141-146 (2008).
5. Lowry, W. E. et al. Generation of human induced pluripotent stem cells from dermal fibroblasts. Proc Natl Acad Sci USA 105, 2883-2888 (2008).
6. Park, I. H. et al. Disease-specific induced pluripotent stem cells. Cell 134, 877-886 (2008).
7. Dimos, J. T. et al. Induced pluripotent stem cells generated from patients with ALS can be differentiated into motor neurons. Science 321, 1218-1221 (2008).
8. Ebert, A. D. et al. Induced pluripotent stem cells from a spinal muscular atrophy patient. Nature (2008).
9. Tischkowitz, M. D. & Hodgson, S. V. Fanconi anaemia. J Med Genet. 40, 1-10 (2003).
10. Wang, W. Emergence of a DNA-damage response network consisting of Fanconi anaemia and BRCA proteins. Nat Rev Genet. 8, 735-748 (2007).
11. Auerbach, A. D. & Wolman, S. R. Susceptibility of Fanconi's anaemia fibroblasts to chromosome damage by carcinogens. Nature 261, 494-496 (1976).
12. Kutler, D. I. et al. A 20-year perspective on the International Fanconi Anemia Registry (IFAR). Blood 101, 1249-1256 (2003).
13. Guardiola, P. et al. Outcome of 69 allogeneic stem cell transplantations for Fanconi anemia using HLA-matched unrelated donors: a study on behalf of the European Group for Blood and Marrow Transplantation. Blood 95, 422-429 (2000).
14. Wagner, J. E. et al. Unrelated donor bone marrow transplantation for the treatment of Fanconi anemia. Blood 109, 2256-2262 (2007).
15. Liu, J. M. et al. Engraftment of hematopoietic progenitor cells transduced with the Fanconi anemia group C gene (FANCC). Hum Gene Ther 10, 2337-2346 (1999).
16. Kelly, P. F. et al. Stem cell collection and gene transfer in Fanconi anemia. Mol Ther 15, 211-219 (2007).
17. Larghero, J. et al. Hematopoietic progenitor cel harvest and functionality in Fanconi anemia patients. Blood 100, 3051 (2002).
18. Jacome, A. et al. A simplified approach to improve the efficiency and safety of ex vivo hematopoietic gene therapy in fanconi anemia patients. Hum Gene Ther 17, 245-250 (2006).
19. Ying, Q. L. et al. The ground state of embryonic stem cell self-renewal. Nature 453, 519-523 (2008).
20. Taniguchi, T. & D'Andrea, A. D. Molecular pathogenesis of Fanconi anemia: recent progress. Blood 107, 4223-4233 (2006).
21. Almarza, E. et al. Characteristics of lentiviral vectors harboring the proximal promoter of the vav proto-oncogene: a weak and efficient promoter for gene therapy. Mol Ther 15, 1487-1494 (2007).
22. Aasen, T. et al. Efficient and rapid generation of induced pluripotent stem cells from human keratinocytes. Nat Biotechnol 26, 1276-1284 (2008).
23. Kalb, R. et al. Hypomorphic mutations in the gene encoding a key Fanconi anemia protein, FANCD2, sustain a significant group of FA-D2 patients with severe phenotype. Am J Hum Genet. 80, 895-910 (2007).
24. Brivanlou, A. H. et al. Stem cells. Setting standards for human embryonic stem cells. Science 300, 913-916 (2003).
25. Raya, A. et al. Generation of cardiomyocytes from new human embryonic stem cell lines derived from poor-quality blastocysts. Cold Spring Harb Symp Quant Biol 73, In press (2008).
26. Pfeifer, A., Ikawa, M., Dayn, Y. & Verma, I. M. Transgenesis by lentiviral vectors: lack of gene silencing in mammalian embryonic stem cells and preimplantation embryos. Proc Natl Acad Sci USA 99, 2140-2145 (2002).
27. Xia, X., Zhang, Y., Zieth, C. R. & Zhang, S. C. Transgenes delivered by lentiviral vector are suppressed in human embryonic stem cells in a promoter-dependent manner. Stem Cells Dev 16, 167-176 (2007).
28. Bogliolo, M. et al. Histone H2AX and Fanconi anemia FANCD2 function in the same pathway to maintain chromosome stability. Embo J26, 1340-1351 (2007).
29. Waisfisz, Q. et al. Spontaneous functional correction of homozygous fanconi anaemia alleles reveals novel mechanistic basis for reverse mosaicism. Nat Genet. 22, 379-383 (1999).
30. Gregory, J. J., Jr. et al. Somatic mosaicism in Fanconi anemia: evidence of genotypic reversion in lymphohematopoietic stem cells. Proc Natl Acad Sci US A 98, 2532-2537 (2001).
31. Gross, M. et al. Reverse mosaicism in Fanconi anemia: natural gene therapy via molecular self-correction. Cytogenet Genome Res 98, 126-135 (2002).
32. Rio, P. et al. In vivo proliferation advantage of genetically corrected hematopoietic stem cells in a mouse model of Fanconi anemia FA-D1. Blood 112, 4853-4861 (2008).
33. Nakano, T., Kodama, H. & Honjo, T. Generation of lymphohematopoietic cells from embryonic stem cells in culture. Science 265, 1098-1101 (1994).
34. Vodyanik, M. A., Bork, J. A., Thomson, J. A. & Slukvin, II. Human embryonic stem cell-derived CD34+ cells: efficient production in the coculture with OP9 stromal cells and analysis of lymphohematopoietic potential. Blood 105, 617-626 (2005).
35. Ji, J., Vijayaragavan, K., Bosse, M., Weisel, K. & Bhatia, M. OP9 stroma augments survival of hematopoietic precursors and progenitors during hematopoietic differentiation from human embryonic stem cells. Stem Cells 26, 2485-2495 (2008).
36. Okita, K., Ichisaka, T. & Yamanaka, S. Generation of germline-competent induced pluripotent stem cells. Nature 448, 313-317 (2007).
37. Nakagawa, M. et al. Generation of induced pluripotent stem cells without Myc from mouse and human fibroblasts. Nat Biotechnol 26, 101-106 (2008).
38. Okita, K., Nakagawa, M., Hyenjong, H., Ichisaka, T. & Yamanaka, S. Generation of mouse induced pluripotent stem cells without viral vectors. Science 322, 949-953 (2008).
39. Stadtfeld, M., Nagaya, M., Utikal, J., Weir, G. & Hochedlinger, K. Induced pluripotent stem cells generated without viral integration. Science 322, 945-949 (2008).
40. Hacein-Bey-Abina, S. et al. Insertional oncogenesis in 4 patients after retrovirus-mediated gene therapy of SCID-X1. J Clin Invest 118, 3132-3142 (2008).
41. Zwaka, T. P. & Thomson, J. A. Homologous recombination in human embryonic stem cells. Nat Biotechnol 21, 319-321 (2003).
42. Casado, J. A. et al. A comprehensive strategy for the subtyping of patients with Fanconi anaemia: conclusions from the Spanish Fanconi Anemia Research Network. J Med Genet. 44, 241-249 (2007).
43. Gonzalez-Murillo, A., Lozano, M. L., Montini, E., Bueren, J. A. & Guenechea, G. Unaltered repopulation properties of mouse hematopoietic stem cells transduced with lentiviral vectors. Blood 112, 3138-3147 (2008).
44. Nijman, S. M. et al. The deubiquitinating enzyme USP1 regulates the Fanconi anemia pathway. Mol Cell 17, 331-339 (2005).
45. Bruun, D. et al. siRNA depletion of BRCA1, but not BRCA2, causes increased genome instability in Fanconi anemia cells. DNA Repair (Amst) 2, 1007-1013 (2003).
46. Takahashi, K. et al. Induction of pluripotent stem cells from adult human fibroblasts by defined factors. Cell 131, 861-872 (2007).
47. Aasen, T. et al. Efficient and rapid generation of induced pluripotent stem cells from human keratinocytes. Nat Biotechnol 26, 1276-1284 (2008).
48. Burdon, T., Stracey, C., Chambers, I., Nichols, J. & Smith, A. Suppression of SHP-2 and ERK signalling promotes self-renewal of mouse embryonic stem cells. Dev Biol 210, 30-43 (1999).
49. Lodge, P., McWhir, J., Gallagher, E. & Sang, H. Increased gp130 signaling in combination with inhibition of the MEK/ERK pathway facilitates embryonic stem cell isolation from normally refractory murine CBA blastocysts. Cloning Stem Cells 7, 2-7 (2005).
50. Sato, N., Meijer, L., Skaltsounis, L., Greengard, P. & Brivanlou, A. H. Maintenance of pluripotency in human and mouse embryonic stem cells through activation of Wnt signaling by a pharmacological GSK-3-specific inhibitor. Nat Med 10, 55-63 (2004).
51. Umehara, H. et al. Efficient derivation of embryonic stem cells by inhibition of glycogen synthase kinase-3. Stem Cells 25, 2705-2711 (2007).
52. Watanabe, K. et al. A ROCK inhibitor permits survival of dissociated human embryonic stem cells. Nat Biotechnol 25, 681-686 (2007).
53. Ying, Q. L. et al. The ground state of embryonic stem cell self-renewal. Nature 453, 519-523 (2008).

What is claimed is:

1. A method for preparing a genetically corrected induced pluripotent stem cell comprising:
(i) transfecting a genetically diseased non-pluripotent cell with a nucleic acid encoding a disease-correcting gene to form a genetically corrected non-pluripotent cell;
(ii) transfecting said genetically corrected non-pluripotent cell with a nucleic acid encoding an OCT4 protein, a nucleic acid encoding a SOX2 protein, a nucleic acid encoding a KLF4 protein and a nucleic acid encoding a cMYC protein to form a genetically corrected transfected non-pluripotent cell; and
(iii) allowing said genetically corrected transfected non-pluripotent cell to divide thereby forming said genetically corrected induced pluripotent stem cell.

2. The method of claim 1, wherein said genetically diseased non-pluripotent cell is a human cell.

3. The method of claim 1, wherein said genetically diseased non-pluripotent cell is a mouse cell.

4. The method of claim 1, wherein said disease-correcting gene encodes a FANCA protein.

5. The method of claim 1, wherein said disease-correcting gene encodes a FANCD2 protein.

6. The method of claim 1, wherein said method further comprises introducing to said genetically corrected transfected non-pluripotent cell of step (iii) at least one kinase inhibitor.

7. The method of claim 1, wherein said method further comprises introducing to said genetically corrected transfected non-pluripotent cell of step (iii) a MEK1 and a GSK3 kinase inhibitor.

8. A method for preparing a genetically corrected induced pluripotent stem cell comprising:
(i) transfecting a genetically diseased non-pluripotent cell with a nucleic acid encoding an OCT4 protein, a nucleic acid encoding a SOX2 protein, a nucleic acid encoding a KLF4 protein and a nucleic acid encoding a cMYC protein to form a transfected genetically diseased non-pluripotent cell;
(ii) allowing said transfected genetically diseased non-pluripotent cell to divide thereby forming a genetically diseased induced pluripotent stem cell; and
(iii) transfecting said genetically diseased induced pluripotent stem cell with a nucleic acid encoding a disease-correcting gene to form said genetically corrected induced pluripotent stem cell.

9. The method of claim 8, wherein said method further comprises introducing to said transfected genetically diseased non-pluripotent cell of step (ii) at least one kinase inhibitor.

10. The method of claim 8, wherein said method further comprises introducing to said transfected genetically diseased non-pluripotent cell of step (ii) a MEK1 and a GSK3 kinase inhibitor.

11. A genetically corrected induced pluripotent stem cell prepared in accordance with the method of either of claim 1 or 8.

12. A method for producing a genetically corrected somatic cell from a genetically diseased mammal comprising:
(a) contacting a genetically corrected induced pluripotent stem cell with cellular growth factors; and
(b) allowing said genetically corrected induced pluripotent stem cell to divide, thereby forming said genetically corrected somatic cell.

13. The method of claim 12, wherein said genetically corrected induced pluripotent stem cell is prepared in accordance with a method comprising:
(i) transfecting a genetically diseased non-pluripotent cell with a nucleic acid encoding a disease-correcting gene to form a genetically corrected non-pluripotent cell;
(ii) transfecting said genetically corrected non-pluripotent cell with a nucleic acid encoding an OCT4 protein, a nucleic acid encoding a SOX2 protein, a nucleic acid encoding a KLF4 protein and a nucleic acid encoding a cMYC protein to form a genetically corrected transfected non-pluripotent cell; and
(iii) allowing said genetically corrected transfected non-pluripotent cell to divide thereby forming said genetically corrected induced pluripotent stem cell.

14. The method of claim 13, wherein said method further comprises introducing to said genetically corrected transfected non-pluripotent cell of step (iii) a kinase inhibitor.

15. The method of claim 13, wherein said method further comprises introducing to said genetically corrected transfected non-pluripotent cell of step (iii) a MEK1 and a GSK3 kinase inhibitor.

16. The method of claim 12, wherein said genetically corrected induced pluripotent stem cell is prepared in accordance with a method comprising:
(i) transfecting a genetically diseased non-pluripotent cell with a nucleic acid encoding an OCT4 protein, a nucleic acid encoding a SOX2 protein, a nucleic acid encoding a KLF4 protein and a nucleic acid encoding a cMYC protein to form a transfected genetically diseased non-pluripotent cell;
(ii) allowing said transfected genetically diseased non-pluripotent cell to divide thereby forming a genetically diseased induced pluripotent stem cell; and
(iii) transfecting said genetically diseased induced pluripotent stem cell with a nucleic acid encoding a disease-correcting gene to form said genetically corrected induced pluripotent stem cell.

17. The method of claim 16, wherein said method further comprises introducing to said transfected genetically diseased non-pluripotent cell of step (ii) at least one kinase inhibitor.

18. The method of claim 16, wherein said method further comprises introducing to said transfected genetically diseased non-pluripotent cell of step (ii) a MEK1 and a GSK3 kinase inhibitor.

19. A method of treating a mammal in need of tissue repair comprising:
(i) administering a genetically corrected induced pluripotent stem cell to said mammal,
(ii) allowing said genetically corrected induced pluripotent stem cell to divide and differentiate into somatic cells in said mammal, thereby providing tissue repair in said mammal.

20. The method of claim 19, wherein said genetically corrected induced pluripotent stem cell is prepared in accordance with a method comprising:
(i) transfecting a genetically diseased non-pluripotent cell with a nucleic acid encoding a disease-correcting gene to form a genetically corrected non-pluripotent cell;
(ii) transfecting said genetically corrected non-pluripotent cell with a nucleic acid encoding an OCT4 protein, a nucleic acid encoding a SOX2 protein, a nucleic acid encoding a KLF4 protein and a nucleic acid encoding a cMYC protein to form a genetically corrected transfected non-pluripotent cell; and
(iii) allowing said genetically corrected transfected non-pluripotent cell to divide thereby forming said genetically corrected induced pluripotent stem cell.

21. The method of claim 20, wherein said method further comprises introducing to said genetically corrected transfected non-pluripotent cell of step (iii) a kinase inhibitor.

22. The method of claim 20, wherein said method further comprises introducing to said transfected genetically corrected non-pluripotent cell of step (iii) a MEK1 and a GSK3 kinase inhibitor.

23. The method of claim 19, wherein said genetically corrected induced pluripotent stem cell is prepared in accordance with a method comprising:
(i) transfecting a genetically diseased non-pluripotent cell with a nucleic acid encoding an OCT4 protein, a nucleic acid encoding a SOX2 protein, a nucleic acid encoding a KLF4 protein and a nucleic acid encoding a cMYC protein to form a transfected genetically diseased non-pluripotent cell;
(ii) allowing said transfected genetically diseased non-pluripotent cell to divide thereby forming a genetically diseased induced pluripotent stem cell; and
(iii) transfecting said genetically diseased induced pluripotent stem cell with a nucleic acid encoding a disease-correcting gene to form said genetically corrected induced pluripotent stem cell.

24. The method of claim 23, wherein said method further comprises introducing to said transfected genetically diseased non-pluripotent cell of step (ii) at least one kinase inhibitor.

25. The method of claim 23, wherein said method further comprises introducing to said transfected genetically diseased non-pluripotent cell of step (ii) a MEK1 and a GSK3 kinase inhibitor.

26. A genetically diseased non-pluripotent cell comprising a nucleic acid encoding a disease-correcting gene, a nucleic acid encoding an OCT4 protein, a nucleic acid encoding a SOX2 protein, a nucleic acid encoding a KLF4 protein and a nucleic acid encoding a cMYC protein.

27. The genetically diseased non-pluripotent cell of claim 26, further comprising at least one kinase inhibitor.

28. The genetically diseased non-pluripotent cell of claim 26, further comprising a Mek1 and a GSK3 inhibitor.

29. The genetically diseased non-pluripotent cell of claim 26, wherein said disease-correcting gene is encoding a FANCA protein.

30. The genetically diseased non-pluripotent cell of claim 26, wherein said disease-correcting gene is encoding a FANC2D protein.


SEQUENCE LISTING (19 sequences)

SEQ ID NO: 1 - protein, 360 aa, homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 2 - protein, 265 aa, homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - protein, 190 aa, homo sapiens
MGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 4 - protein, 317 aa, homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - protein, 479 aa, homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 6 - protein, 454 aa, homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 7 - protein, 1455 aa, homo sapiens
MSDSWVPNSASGQDPGGRRRAWAELLAGRVKREKYNPERAQKLKESAVRLLRSHQDLNALLLEVEGPLCKKLSLSKVIDCDSSEAYANHSSSFIGSALQDQASRLGVPVGILSAGMVASSVGQICTAPAETSHPVLLTVEQRKKLSSLLEFAQYLLAHSMFSRLSFCQELWKIQSSLLLEAVWHLHVQGIVSLQELLESHPDMHAVGSWLFRNLCCLCEQMEASCQHADVARAMLSDFVQMFVLRGFQKNSDLRRTVEPEKMPQVTVDVLQRMLIFALDALAAGVQEESSTHKIVRCWFGVFSGHTLGSVISTDPLKRFFSHTLTQILTHSPVLKASDAVQMQREWSFARTHPLLTSLYRRLFVMLSAEELVGHLQEVLETQEVHWQRVLSFVSALVVCFPEAQQLLEDWVARLMAQAFESCQLDSMVTAFLVVRQAALEGPSAFLSYADWFKASFGSTRGYHGCSKKALVFLFTFLSELVPFESPRYLQVHILHPPLVPGKYRSLLTDYISLAKTRLADLKVSIENMGLYEDLSSAGDITEPHSQALQDVEKAIMVFEHTGNIPVTVMEASIFRRPYYVSHFLPALLTPRVLPKVPDSRVAFIESLKRADKIPPSLYSTYCQACSAAEEKPEDAALGVRAEPNSAEEPLGQLTAALGELRASMTDPSQRDVISAQVAVISERLRAVLGHNEDDSSVEISKIQLSINTPRLEPREHMAVDLLLTSFCQNLMAASSVAPPERQGPWAALFVRTMCGRVLPAVLTRLCQLLRHQGPSLSAPHVLGLAALAVHLGESRSALPEVDVGPPAPGAGLPVPALFDSLLTCRTRDSLFFCLKFCTAAISYSLCKFSSQSRDTLCSCLSPGLIKKFQFLMFRLFSEARQPLSEEDVASLSWRPLHLPSADWQRAALSLWTHRTFREVLKEEDVHLTYQDWLHLELEIQPEADALSDTERQDFHQWAIHEHFLPESSASGGCDGDLQAACTILVNALMDFHQSSRSYDHSENSDLVFGGRTGNEDIISRLQEMVADLELQQDLIVPLGHTPSQEHFLFEIFRRRLQALTSGWSVAASLQRQRELLMYKRILLRLPSSVLCGSSFQAEQPITARCEQFFHLVNSEMRNFCSHGGALTQDITAHFFRGLLNACLRSRDPSLMVDFILAKCQTKCPLILTSALVWWPSLEPVLLCRWRRHCQSPLPRELQKLQEGRQFASDFLSPEAASPAPNPDWLSAAALHFAIQQVREENIRKQLKKLDCEREELLVFLFFFSLMGLLSSHLTSNSTTDLPKAFHVCAAILECLEKRKISWLALFQLTESDLRLGRLLLRVAPDQHTRLLPFAFYSLLSYFHEDAAIREEAFLHVAVDMYLKLVQLFVAGDTSTVSPPAGRSLELKGQGNPVELITKARLFLLQLIPRCPKKSFSHVAELLADRGDCDPEVSAALQSRQQAAPDADLSQEPHLF

SEQ ID NO: 8 - protein, 1471 aa, homo sapiens
MVSKRRLSKSEDKESLTEDASKTRKQPLSKKTKKSHIANEVEENDSIFVKLLKISGIILKTGESQNQLAVDQIAFQKKLFQTLRRHPSYPKIIEEFVSGLESYIEDEDSFRNCLLSCERLQDEEASMGASYSKSLIKLLLGIDILQPAIIKTLFEKLPEYFFENKNSDEINIPRLIVSQLKWLDRVVDGKDLTTKIMQLISIAPENLQHDIITSLPEILGDSQHADVGKELSDLLIENTSLTVPILDVLSSLRLDPNFLLKVRQLVMDKLSSIRLEDLPVIIKFILHSVTAMDTLEVISELREKLDLQHCVLPSRLQASQVKLKSKGRASSSGNQESSGQSCIILLFDVIKSAIRYEKTISEAWIKAIENTASVSEHKVFDLVMLFIIYSTNTQTKKYIDRVLRNKIRSGCIQEQLLQSTFSVHYLVLKDMCSSILSLAQSLLHSLDQSIISFGSLLYKYAFKFFDTYCQQEVVGALVTHICSGNEAEVDTALDVLLELVVLNPSAMMMNAVFVKGILDYLDNISPQQIRKLFYVLSTLAFSKQNEASSHIQDDMHLVIRKQLSSTVFKYKLIGIIGAVTMAGIMAADRSESPSLTQERANLSDEQCTQVTSLLQLVHSCSEQSPQASALYYDEFANLIQHEKLDPKALEWVGHTICNDFQDAFVVDSCVVPEGDFPFPVKALYGLEEYDTQDGIAINLLPLLFSQDFAKDGGPVTSQESGQKLVSPLCLAPYFRLLRLCVERQHNGNLEEIDGLLDCPIFLTDLEPGEKLESMSAKERSFMCSLIFLTLNWFREIVNAFCQETSPEMKGKVLTRLKHIVELQIILEKYLAVTPDYVPPLGNFDVETLDITPHTVTAISAKIRKKGKIERKQKTDGSKTSSSDTLSEEKNSECDPTPSHRGQLNKEFTGKEEKTSLLLHNSHAFFRELDIEVFSILHCGLVTKFILDTEMHTEATEVVQLGPPELLFLLEDLSQKLESMLTPPIARRVPFLKNKGSRNIGFSHLQQRSAQEIVHCVFQLLTPMCNHLENIHNYFQCLAAENHGVVDGPGVKVQEYHIMSSCYQRLLQIFHGLFAWSGFSQPENQNLLYSALHVLSSRLKQGEHSQPLEELLSQSVHYLQNFHQSIPSFQCALYLIRLLMVILEKSTASAQNKEKIASLARQFLCRVWPSGDKEKSNISNDQLHALLCIYLEHTESILKAIEEIAGVGVPELINSPKDASSSTFPTLTRHTFVVFFRVMMAELEKTVKKIEPGTAADSQQIHEEKLLYWNMAVRDFSILINLIKVFDSHPVLHVCLKYGRLFVEAFLKQCMPLLDFSFRKHREDVLSLLETFQLDTRLLHHLCGHSKIHQDTRLTQHVPLLKKTLELLVCRVKAMLTLNNCREAFWLGNLKNRDLQGEEIKSQNSQESTADESEDDMSSQASKSKATEVSLQNPPESGTDGCILLIVLSWWSRTLPTYVYCQMLLCPFPFPP

SEQ ID NO: 9 - DNA, 19 bp, Artificial
  Synthetic DNA primer for FANCA transgene.
gctcaagggtcagggcaac

SEQ ID NO: 10 - DNA, 21 bp, Artificial
  Synthetic DNA primer for FNACA transgene.
tgtgagaagctctttttcggg

SEQ ID NO: 11 - DNA, 31 bp, Artificial
  Synthetic DNA probe to quantify proviral copy number.
cgtctttttctgctgcagttaatacctcggt

SEQ ID NO: 12 - DNA, 19 bp, Artificial
  Synthetic DNA primer to quantify number of cells.
attggcaatgagcggttcc

SEQ ID NO: 13 - DNA, 21 bp, Artificial
  Synthetic DNA primer.
acagtctccactcacccagga

SEQ ID NO: 14 - DNA, 25 bp, Artificial
  Synthetic DNA probe.
ccctgaggcactcttccagccttcc

SEQ ID NO: 15 - DNA, 19 bp, Artificial
  Synthetic DNA primer.
tcttctgacgggacctgcc

SEQ ID NO: 16 - DNA, 29 bp, Artificial
  Synthetic DNA primer
aagagctccatgttatgcttgtaataaat

SEQ ID NO: 17 - DNA, 24 bp, Artificial
  Synthetic DNA probe.
cacaccagcccagctcccgtgtaa

SEQ ID NO: 18 - DNA, 18 bp, Artificial
  Synthetic DNA primer.
cacaggactccatgccca

SEQ ID NO: 19 - DNA, 21 bp, Artificial
  Synthetic Luciferase siRNA having deoxythymidine at positions 20-21.
cguacgcggaauacuucgatt